(19) United States
(12) Patent Application Publication
(10) Pub. No.: US 2004/0121460 A1
(43) Pub. Date: Jun. 24, 2004

(54) Differentiation of stem cells to pancreatic endocrine cells

(76) Inventors: Nadya L Lumelsky, Washington, DC (US); Oliver Blondel, Bethesda, MD (US); Ronald D McKay, Bethesda, MD (US); Jong-Hoon Kim, Rockville, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/470,030

(22) PCT Filed: Jan. 24, 2002
(86) PCT No.: PCT/US02/02361

(51) Int. Cl. C12N 5/06; C12N 5/08

(52) U.S. Cl. 435/366; 435/354

(57) ABSTRACT

A method is provided for differentiating embryonic stem cells to endocrine cells. The method includes generating embryoid bodies from a culture of undifferentiated embryonic stem cells, selecting endocrine precursor cells, expanding the endocrine precursor cells by culturing endocrine cells in an expansion medium that comprises a growth factor, and differentiating the expanded endocrine precursor cells in a differentiation media to differentiated endocrine cells produced by this method are also provided. Artificial islets are disclosed, as well as method for using the pancreatic endocrine cells and the artificial islets.

### FIELD OF THE INVENTION

This invention relates to the field of the treatment of diabetes, more specifically to the production of in vitro models of the islet of Langerhans, and to the production of insulin-producing cells.

### BACKGROUND OF THE INVENTION

A mammalian pancreas is composed of two subclasses of tissue: the exocrine cells of the acinar tissue and the endocrine cells of the islets of Langerhans. The exocrine cells produce the digestive enzymes which are secreted through the pancreatic duct to the intestine. The islet cells produce the polypeptide hormones which are involved in carbohydrate metabolism. The islands of endocrine tissue that exist within the adult mammalian pancreas are termed the islets of Langerhans. Adult mammalian islets are composed of four major cell types, the α, β, δ, and PP cells, which produce glucagon, insulin, somatostatin, and pancreatic polypeptide, respectively.

Diabetes is defined as a failure of cells to transport endogenous glucose across their membranes either because of an endogenous deficiency of insulin or an insulin receptor defect. Diabetes type I, or insulin dependent diabetes mellitis (IDDM) is caused by the destruction of β cells, which results in insufficient levels of endogenous insulin. Diabetes type II, or non-insulin dependent diabetes, is believed to be a defect in either the insulin receptor itself or in the number of insulin receptors present or in the balance between insulin and glucagon signals. Although diabetes runs in families, and it appears that genetics is involved in the development of the disease, no one genetic marker has been identified that is responsible for this condition.

Current treatment of individuals with clinical manifestation of diabetes attempts to emulate the role of the pancreatic β cells in a non-diabetic individual. Individuals with normal β cell function have tight regulation of the amount of insulin secreted into their bloodstream. This regulation is due to a feed-back mechanism that resides in the β cells that ordinarily prevents surges of blood sugar outside of the normal limits. Unless blood sugar is controlled properly, dangerous, even fatal, levels can result. Hence, treatment of a diabetic individual involves the use of injected bovine, porcine, or cloned human insulin on a daily basis.

Injected insulin and diet regulation permit survival and in many cases a good quality of life for years after onset of the disease. However, there is often a gradual decline in the health of diabetics that has been attributed to damage to the vascular system due to the inevitable surges (both high and low) in the concentration of glucose in the blood of diabetic patients. In short, diabetics treated with injected insulin cannot adjust their intake of carbohydrates and injection of insulin with sufficient precision of quantity and timing to prevent temporary surges of glucose outside of normal limits. These surges are believed to result in various vascular and microvascular disorders that impair normal visual, renal, and even ambulatory functions.

Both of these disease states, i.e., type I and type II diabetes, involve millions of people in the United States alone. Clearly, there is a need to provide a good iii vitro model of the Islet of Langerhans, in order to study the disease process and to investigate new potential therapies. In addition, there is a need to produce new treatments for diabetes, including the production of islet cells for transplantation (see U.S. Pat. No. 4,439,521; U.S. Pat. No. 5,510,263; U.S. Pat. No. 5,646,035; U.S. Pat. No. 5,961,972). Successful transplants of whole isolated islets, for example, have been made in animals and in humans. However, long term resolution of diabetic symptoms has not yet been achieved by this method (Robertson, New England J. Med., 327:1861-1863,1992). There is a need to produce large quantities of islet cells that are autologous, or are not recognized by the immune system.

ES cells can proliferate indefinitely in an undifferentiated state. Furthermore, embryonic stem (ES) cells are totipotent cells, meaning that they can generate all of the cells present in the body (bone, muscle, brain cells, etc.). ES cells have been isolated from the inner cell mass of the developing murine blastocyst (Evans et al., Nature 292:154-156, 1981; Martin et al., Proc. Natl. Acad. Sci. 78:7634-7636, 1981; Robertson et al., Nature 323:445-448, 1986; Doetschman et al., Nature 330:576-578, 1987; and “Thomas et al., Cell 51:503-512, 1987;U.S. Pat. No. 5,670,372). Additionally, human cells with ES properties have recently been isolated from the inner blastocyst cell mass (Thomson et al., Science 282:1145-1147, 1998) and developing germ cells (Shamblott et al., Proc. Natl. Acad. Sci. U.S.A. 95:13726-13731, 1998) (see also U.S. Pat. No. 6,090,622, WO 00/70021 and WO 00/27995).

### SUMMARY OF THE INVENTION

An isolated pancreatic endocrine cell is provided. This cell is differentiated from an embryonic stem cell in vitro.

A method is provided for differentiating embryonic stem cells to endocrine cells. The method includes generating embryoid bodies from a culture of undifferentiated embryonic stem cells, selecting endocrine precursor cells, expanding the endocrine precursor cells by culturing endocrine cells in an expansion medium that comprises a growth factor, and differentiating the expanded endocrine precursor cells in a differentiation medium to differentiated endocrine cells.

A method is also provided for producing an artificial islet. The method includes expanding embryonic stem cells and generating embryoid bodies from a culture of undifferentiated embryonic stem cells, selecting pancreatic endocrine precursor cells, expanding the pancreatic endocrine precursor cells by culturing pancreatic endocrine cells in an expansion medium that includes a growth factor; and differentiating the expanded pancreatic endocrine precursor cells in a differentiation medium to form pancreatic endocrine cells, wherein the differentiation produces an artificial islet. The artificial islets can be transplanted into subjects in need of enhanced islet activity, such as diabetics.

A method is provided for testing an agent to determine the effect of the agent on secretion or expression of a pancreatic hormone by contacting pancreatic endocrine cells to the agent, wherein the pancreatic endocrine cells are differentiated from embryonic stem cells and assaying a parameter of the pancreatic endocrine cell to determine the effect of the agent on the secretion or expression of the pancreatic hormone, or on the extent of differentiation of endocrine cells in the pancreas.

The foregoing and other objects, features, and advantages of the invention will become more apparent from the following detailed description of a several embodiments which proceeds with reference to the accompanying figures.

### BRIEF DESCRIPTION OF THE FIGURES

FIG. 1 is a diagram of one protocol for the differentiation of ES cells to pancreatic endocrine cells.

FIG. 2 is a digital image showing insulin-producing cells differentiated from embryonic stem cells contain different hormone-producing cell types and are organized in three-dimensional clusters with topological organization of pancreatic islets. FIG. 2A shows an inner core of insulin cells (grey) surrounded by an outer layer of glucagon producing cells (white). FIG. 1B is a digital image showing an inner core of insulin producing cells (grey) surrounded by an outer layer of somatostain producing cells (white).

FIG. 3 is a set of graphs and figures demonstrating that islet clusters release insulin in response to glucose utilizing normal pancreatic mechanisms. FIG. 3A is a graph of insulin release in response to different glucose concentrations. Exposure to 50 mM sucrose was used to test for a potential effect of high osmolarity on insulin release. FIG. 3B is a diagrammatic summary of the documented actions of glucose, cAMP, K⁺ and Ca²⁺ on insulin secretion. Effects of known pharmacological regulators of insulin release are indicated. DAG, diacylglycerol; PKA, protein kinase A; PKC, protein kinase C; PLC, phospholipase C. FIG. 3C is a schematic diagram of insulin release in response to various secretaguogues in the presence of 5 mM of glucose. FIG. 3D is a set of bar graphs showing insulin release in response to 20 MM glucose in the presence or absence of inhibitors of insulin secretion.

FIG. 4 is a diagram of the differentiation of pancreatic endocrine cells from pancreatic endocrine stem cells to differentiated α cells, β cells, δ cells, and PP cells.

FIG. 5 is a set of panels showing the neural and pancreatic differentiation of ES cells. FIG. 1A is a set of digital images showing the cells during the procedure for induction of midbrain dopaminergic neurons from ES cells as previous described (see WO 01/83715, herein incorporated by reference). Briefly, the ES cells were taken through 5 steps or stages. In stage 1 undifferentiated ES cells were cultured for 5 days in the presence of 15% fetal calf serum (FCS) on gelatin coated tissue culture dishes in the presence of LIF (1,400 U/ml). In stage 2 embryoid bodies (Ebs) were generated in the presence of FCS for 4 days in the presence or absence of LIF (1,000 U/ml.). In stage 3, the EBs were plated into ITSFn medium (Okabe et al., Mech. Dev. 59: 89-102, 1996) where over 10 days Nestin+ cells migrated from the cell aggregates. In stage 4 these Nestin+ cells were resuspended and expanded for 4 days in N2 medium containing bFGF, sonic hedgehog (Shh) and fibroblast growth factor-8 (FGF8). In stage 5 the medium was changed into N2 medium without bFGF, Shh or FGF8. These cells differentiated efficiently into neurons and astrocytes over a two week period. Embryoid bodies were generated in the presence (LIF+) or absence (LIF−) of LIF (1000 U/ml) and differentiated. Double-immunostaining for TuJ1/GFAP (upper panels, day 8 in stage 5) and PDX-1/En-1 (lower panels, day 3 in stage 4). LIF treatment in stage 2 (EB formation) increases the neuronal (TuJ1+cells, light grey) and decreases the astrocytic (GFAP+, dark grey) population. LIF treatment efficiently enhances midbrain precursor cells (En-1+ cells, dark grey) and negatively regulates pancreatic precursor cells (PDX-1+cells, light grey). FIG. 5C is a bar graph showing that the yield of En-1+ and PDX-1+ cells is expressed as a percentage of total cells at stage 4.

### DETAILED DESCRIPTION OF SEVERAL EMBODIMENTS

The following definitions and methods are provided to better define the present invention and to guide those of ordinary skill in the art in the practice of the present invention. Definitions of common terms may also be found in Rieger et al., Glossary of Genetics: Classical and Molecular, 5th edition, Springer-Verlag: New York, 1991; and Lewin, Genes V, Oxford University Press: New York, 1994. The standard one- and three letter nomenclature for amino acid residues is used.

Additional definitions of terms commonly used in molecular genetics can be found in Benjamin Lewin, Genes V published by Oxford University Press, 1994 (ISBN 0-19-854287-9); Kendrew et al (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

### Terms

α cells are mature glucagon producing cells. In vivo, these cells are found in the pancreatic islets of Langerhans.

β cells are mature insulin producing cells. In vivo, these cells are found in the pancreatic islets of Langerhans,

δ cells are the mature somatostatin producing cells. In vivo, these cells are found in the pancreatic islets of Langerhans.

PP cells are the mature pancreatic polypeptide (PP) producing cells. In vivo, these cells are found in the pancreatic islets of Langerhans.

Animal: Living multi-cellular vertebrate organisms, a category that includes, for example, mammals and birds. The term mammal includes both human and non-human mammals. Similarly, the term “subject” includes both human and veterinary subjects.

Artificial Islets are clusters of pancreatic endocrine cells formed by the differentiation of ES cell in vitro, dislodged clusters of pancreatic endocrine cells differentiated from ES cells in vitro, or by aggregating pancreatic endocrine cells in vitro.

Differentiation refers to the process whereby relatively unspecialized cells (e.g., embryonic cells) acquire specialized structural and/or functional features characteristic of mature cells. Similarly, “differentiate” refers to this process. Typically, during differentiation, cellular structure alters and tissue-specific proteins appear. The term “differentiated pancreatic endocrine cell” refers to cells expressing a protein characteristic of the specific pancreatic endocrine cell type. A differentiated pancreatic endocrine cell includes an α cell, a β cell, a δ cell, and a PP cell, which express glucagon, insulin, somatostatin, and pancreatic polypeptide, respectively.

Differentiation Medium is a synthetic set of culture conditions with the nutrients necessary to support the growth or survival of microorganisms or culture cells, and which allows the differentiation of stem cells into differentiated cells.

Growth factor: a substance that promotes cell growth, survival, and/or differentiation. Growth factors include molecules that function as growth stimulators (mitogens), molecules that function as growth inhibitors (e.g. negative growth factors) factors that stimulate cell migration, factors that function as chemotactic agents or inhibit cell migration or invasion of tumor cells, factors that modulate differentiated functions of cells, factors involved in apoptosis, or factors that promote survival of cells without influencing growth and differentiation. Examples of growth factors are bFGF, EGF, CNTF, HGF, NGF, and actvin-A.

Growth medium or exapansion medium is synthetic set of culture conditions with the nutrients necessary to support the growth (expansion) of a specific population of cells. In one embodiment, the cells are ES cells. In this embodiment, the growth media is an ES growth medium that allows ES cells to proliferate. In another embodiment, the cells are pancreatic endocrine precursor cells. In this embodiment, the expansion medium is a pancreatic endocrine precursor cell expansion medium that allows pancreatic endocrine cell precursors to proliferate.

Growth media generally include a carbon source, a nitrogen source and a buffer to maintain pH. In one embodiment, ES growth medium contains a minimal essential media, such as DMEM, supplemented with various nutrients to enhance ES cell growth. Additionally, the minimal essential media may be supplemented with additives such as horse, calf or fetal bovine serum

Effective amount or Therapeutically effective amount is the amount of agent is an sufficient to prevent, treat, reduce and/or ameliorate the symptoms and/or underlying causes of any of a disorder or disease. In one embodiment, an “effective amount” is sufficient to reduce or eliminate a symptom of a disease. In another embodiment, an effective amount is an amount sufficient to overcome the disease itself.

Embryoid bodies are ES cell aggregates generated when ES cells are plated on a non-adhesive surface that prevents attachment and differentiation of the ES cells. Generally, embryoid bodies include an inner core of undifferentiated stem cells surrounded by primitive endoderm.

Embryonic stem (ES) cells are pluripotent cells isolated from the inner cell mass of the developing blastocyst. “ES cells” can be derived from any organism. ES cells can be derived from mammals. In one embodiment, ES cells are produced from mice, rats, rabbits, guinea pigs, goats, pigs, cows and humans. Human and murine derived ES cells are preferred. ES cells are totipotent cells, meaning that they can generate all of the cells present in the body (bone, muscle, brain cells, etc.). Methods for producing murine ES cells can be found in U.S. Pat. No. 5,670,372, herein incorporated by reference. Methods for producing human ES cells can be found in U.S. Pat. No. 6,090,622, WO 00/70021 and WO 00/27995, herein incorporated by reference.

Expand refers to a process by which the number or amount of cells in a cell culture is increased due to cell division. Similarly, the terms “expansion” or “expanded” refers to this process. The terms “proliferate,” “proliferation” or “proliferated” may be used interchangeably with the words “expand,” “expansion”, or “expanded.” Typically, during an expansion phase, the cells do not differentiate to form mature cells.

Fibroblast growth factor or “FGF” refers to any suitable fibroblast growth factor, derived from any animal, and functional fragments thereof. A variety of FGF's are known and include, but are not limited to, FGF-1 (acidic fibroblast growth factor), FGF-2 (basic fibroblast growth factor, bFGF), FGF-3 (int-2), FGF-4 (hst/K-FGF), FGF-5, FGF-6, FGF-7, FGF-8, FGF-9 and FGF-98. “FGF” refers to a fibroblast growth factor protein such as FGF-1, FGF-2, FGF-4, FGF-6, FGF-8, FGF-9 or FGF-98, or a biologically active fragment or mutant thereof. The FGF can be from any animal species. In one embodiment the FGF is mammalian FGF including but not limited to, rodent, avian, canine, bovine, porcine, equine, and human. The amino acid sequences and method for making many of the FGFs are well known in the art.

The amino acid sequence of human FGF-1 and a method for its recombinant expression are disclosed in U.S. Pat. No. 5,604,293. The amino acid sequence of human FGF-2 and methods for its recombinant expression are disclosed in U.S. Pat. No. 5,439,818, herein incorporated by reference. The amino acid sequence of bovine FGF-2 and various methods for its recombinant expression are disclosed in U.S. Pat. No. 5,155,214, also herein incorporated by reference. When the 146 residue forms are compared, their amino acid sequences are nearly identical with only two residues that differ.

The amino acid sequence of FGF-3 (Dickson et al., Nature 326:833, 1987) and human FGF-4 (Yoshida, et al., PHAS USA, 84:7305-7309, 1987) are known. When the amino acid sequences of human FGF-4, FGF-1, FGF-2 and murine FGF-3 are compared, residues 72-204 of human FGF-4 have 43% homology to human FGF-2; residues 79-204 have 38% homology to human FGF-1; and residues 72-174 have 40% homology to murine FGF-3. The cDNA and deduced amino acid sequences for human FGF-5 (Zhan, et al., Molec. And Cell. Biol., 8(8):3487-3495, 1988), human FGF-6 (Coulier et al., Oncogene 6:1437-1444, 1991), human FGF-7 (Miyamoto, et. al., Mol. And Cell. Biol. 13(7):4251-4259, 1993) are also known. The cDNA and deduced amino acid sequence of murine FGF-8 (Tanaka et. A., PNAS USA, 89:8928-8932, 1992), human and murine FGF-9 (Santos-Ocamp, et. al, J. Biol. Chem., 271(3):1726-1731, 1996) and human FGF-98 (provisional patent application Serial No. 60/083,553 which is hereby incorporated herein by reference in its entirety) are also known.

bFGF-2, and other FGFs, can be made as described in U.S. Pat. No. 5,155,214 (“the '214 patent”). The recombinant bFGF-2, and other FGFs, can be purified to pharmaceutical quality (98% or greater purity) using the techniques described in detail in U.S. Pat. No. 4,956,455.

Biologically active variants of FGF are also of use with the methods disclosed herein. Such variants should retain FGF activities, particularly the ability to bind to FGF receptor sites. FGF activity may be measured using standard FGF bioassays, which are known to those of skill in the art. Representative assays include known radioreceptor assays using membranes, a bioassay that measures the ability of the molecule to enhance incorporation of tritiated thymidine, in a dose-dependent manner, into the DNA of cells, and the like. Preferably, the variant has at least the same activity as the native molecule.

In addition to the above described FGFs, an agent of use also includes an active fragment of any one of the above-described FGFs. In its simplest form, the active fragment is made by the removal of the N-terminal methionine, using well-known techniques for N-terminal Met removal, such as a treatment with a methionine aminopeptidase. A second desirable truncation includes an FGF without its leader sequence. Those skilled in the art recognize the leader sequence as the series of hydrophobic residues at the N-terminus of a protein that facilitate its passage through a cell membrane but that are not necessary for activity and that are not found on the mature protein.

Preferred truncations on the FGFs are determined relative to mature FGF-2 having 146 residues. As a general rule, the amino acid sequence of an FGF is aligned with FGF-2 to obtain maximum homology. Portions of the FGF that extend beyond the corresponding N-terminus of the aligned FGF-2 are generally suitable for deletion without adverse effect. Likewise, portions of the FGF that extend beyond the C-terminus of the aligned FGF-2 are also capable of being deleted without adverse effect.

Fragments of FGF that are smaller than those described can also be employed in the present invention.

Suitable biologically active variants can be FGF analogues or derivatives. By “analogue” is intended an analogue of either FGF or an FGF fragment that includes a native FGF sequence and structure having one or more amino acid substitutions, insertions, or deletions. Analogs having one or more peptoid sequences (peptide mimic sequences) are also included (see e.g. International Publication No. WO 91/04282). By “derivative” is intended any suitable modification of FGF, FGF fragments, or their respective analogues, such as glycosylation, phosphorylation, or other addition of foreign moieties, so long as the FGF activity is retained. Methods for making FGF fragments, analogues, and derivatives are available in the art.

In addition to the above-described FGFs, the method of the present invention can also employ an active mutant or variant thereof. By the term active mutant, as used in conjunction with an FGF, is meant a mutated form of the naturally occurring FGF. FGF mutant or variants will generally have at least 70%, preferably 80%, more preferably 85%, even more preferably 90% to 95% or more, and for example 98% or more amino acid sequence identity to the amino acid sequence of the reference FGF molecule. A mutant or variant may, for example, differ by as few as 1 to 10 amino acid residues, such as 6-10, as few as 5, as few as 4, 3, 2, or even 1 amino acid residue.

The sequence identity can be determined as described herein. For FGF, one method for determining sequence identify employs the Smith-Waterman homology search algorithm (Meth. Mol. Biol. 70:173-187 (1997)) as implemented in MSPRCH program (Oxford Molecular) using an affine gap search with the following search parameters: gap open penalty of 12, and gap extension penalty of 1. In one embodiment, the mutations are “conservative amino acid substitutions” using L-amino acids, wherein one amino acid is replaced by another biologically similar amino acid. Conservative amino acid substitutions are those that preserve the general charge, hydrophobicity, hydrophilicity, and/or steric bulk of the amino acid being substituted.

One skilled in the art, using art known techniques, is able to make one or more point mutations in the DNA encoding any of the FGFs to obtain expression of an FGF polypeptide mutant (or fragment mutant) having angiogenic activity for use in methods disclosed herein. To prepare a biologically active mutant of an FGF, one uses standard techniques for site directed mutagenesis, as known in the art and/or as taught in Gilman, et al., Gene, 8:81 (1979) or Roberts, et al., Nature, 328:731 (1987), to introduce one or more point mutations into the cDNA that encodes the FGF.

Heterologous: A heterologous sequence is a sequence that is not normally (i.e. in the wild-type sequence) found adjacent to a second sequence. In one embodiment, the sequence is from a different genetic source, such as a virus or organism, than the second sequence.

Hybridization is the process wherein oligonucleotides and their analogs bind by hydrogen bonding, which includes Watson-Crick, Hoogsteen or reversed Hoogsteen hydrogen bonding, between complementary bases. Generally, nucleic acid consists of nitrogenous bases that are either pyrimidines (Cytosine (C), uracil (U), and thymine (T)) or purines (adenine (A) and guanine (G)). These nitrogenous bases form hydrogen bonds consisting of a pyrimidine bonded to a purine, and the bonding of the pyrimidine to the purine is referred to as “base pairing.” More specifically, A will bond to T or U, and G will bond to C. “Complementary” refers to the base pairing that occurs between two distinct nucleic acid sequences or two distinct regions of the same nucleic acid sequence. For example, a M-CSF antagonist can be an oligonucleotide complementary to a M-CSF encoding mRNA, or a M-CSF encoding dsDNA.

“Specifically hybridizable” and “specifically complementary” are terms which indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions in which specific binding is desired, for example under physiological conditions in the case of in vivo assays. Such binding is referred to as “specific hybridization.”

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization.

Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch. The parameters of salt concentration and temperature can be varied to achieve the optimal level of identity between the probe and the target nucleic acid. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11, herein incorporated by reference.

For purposes of the present invention, “stringent conditions” encompass conditions under which hybridization will only occur if there is less than 30% mismatch between the hybridization molecule and the target sequence. “Stringent conditions” may be broken down into particular levels of stringency for more precise definition. Thus, as used herein, “moderate stringency” conditions are those under which molecules with more than 30% sequence mismatch will not hybridize; conditions of “medium stringency” are those under which molecules with more than 20% mismatch will not hybridize, and conditions of “high stringency” are those under which sequences with more than 10% mismatch will not hybridize.

Molecules with complementary nucleic acids form a stable duplex or triplex when the strands bind, or hybridize, to each other by forming Watson-Crick, Hoogsteen or reverse Hoogsteen base pairs. Stable binding occurs when an oligonucleotide remains detectably bound to a target nucleic acid sequence under the required conditions. “Complementarity” is the degree to which bases in one nucleic acid strand base pair with the bases in a second nucleic acid strand. Complementarity is conveniently described by the percentage, i.e. the proportion of nucleotides that form base pairs between two strands or within a specific region or domain of two strands. For example, if 10 nucleotides of a 15 nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between the oligonucleotide and the target sequence to achieve detectable binding, and disrupt expression of gene products (such as M-CSF). When expressed or measured by percentage of base pairs formed, the percentage complementarity that fulfills this goal can range from as little as about 50% complementarity to full, (100%) complementary. In general, sufficient complementarity is at least about 50%. In one embodiment, sufficient complementarity is at least about 75% complementarity. In another embodiment, sufficient complementarity is at least about 90% or about 95% complementarity. In yet another embodiment, sufficient complementarity is at least about 98% or 100% complementarity.

A thorough treatment of the qualitative and quantitative considerations involved in establishing binding conditions that allow one skilled in the art to design appropriate oligonucleotides for use under the desired conditions is provided by Beltz et al. Methods Enzymol 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Islets of Langerhans are small discrete clusters of pancreatic endocrine tissue. In vivo, in an adult mammal, the islets of Langerhans are found in the pancreas as discrete clusters (islands) of pancreatic endocrine tissue surrounded by the pancreatic exocrine (or ascinar) tissue. In vivo, the islets of Langerhans consist of the α cells, β cells, δ cells, and PP cells. Histologically, the islets of Langerhans consist of a central core of β cells surrounded by an outer layer of α cells, δ cells, and PP cells. The islets of Langerhans are sometimes referred to herein as “islets.”

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

LIF (Leukemia Inhibitory Factor) is a growth factor that prevents differentiation of ES cells. LIF is a heavily and variably glycosylated 58 kDa protein with a length of 179 amino acids. Glycosylation does not appear to be essential for bioactivity. Two different glycosylation variants have been designated as LIF-A and LIF-B. The murine and human factors show a homology of 79 percent at the amino acid level. Both factors show a high degree of conservative amino acid exchanges.

Nucleotide includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Polypeptide refers to a polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those which are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell. An “epitope” is a region of a polypeptide capable of binding an immunoglobulin generated in response to contact with an antigen. Thus, smaller peptides containing the biological activity of insulin, or conservative variants of the insulin, are thus included as being of use.

The term “soluble” refers to a form of a polypeptide that is not inserted into a cell membrane.

The term “substantially purified polypeptide” as used herein refers to a polypeptide which is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative substitutions replace one amino acid with another amino acid that is similar in size, hydrophobicity, etc. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Variations in the cDNA sequence that result in amino acid changes, whether conservative or not, should be minimized in order to preserve the functional and immunologic identity of the encoded protein. The immunologic identity of the protein may be assessed by determining whether it is recognized by an antibody; a variant that is recognized by such an antibody is immunologically conserved. Any cDNA sequence variant will preferably introduce no more than twenty, and preferably fewer than ten amino acid substitutions into the encoded polypeptide. Variant amino acid sequences may, for example, be 80, 90 or even 95% or 98% identical to the native amino acid sequence.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent or “drug” refers to a chemical compound or composition capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Polynucleotide is a nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Primers: Short nucleic acids, for example DNA oligonucleotides 10 nucleotides or more in length, which are annealed to a complementary target DNA strand by nucleic acid hybridization to form a hybrid between the primer and the target DNA strand, then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Probes and primers as used in the present invention may, for example, include at least 10 nucleotides of the nucleic acid sequences that are shown to encode specific proteins. In order to enhance specificity, longer probes and primers may also be employed, such as probes and primers that comprise 15, 20, 30, 40, 50, 60, 70, 80, 90 or 100 consecutive nucleotides of the disclosed nucleic acid sequences. Methods for preparing and using probes and primers are described in the references, for example Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences; Innis et al. (1990) PCR Protocols, A Guide to Methods and Applications, Innis et al. (Eds.), Academic Press, San Diego, Calif. PCR primer pairs can be derived from a known sequence, for example, by using computer programs intended for that purpose such as Primer (Version 0.5, 1991, Whitehead Institute for Biomedical Research, Cambridge, Mass.).

When referring to a probe or primer, the term specific for (a target sequence) indicates that the probe or primer hybridizes under stringent conditions substantially only to the target sequence in a given sample comprising the target sequence.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Similarly, a recombinant protein is one encoded for by a recombinant nucleic acid molecule.

Stem cell refers to a cell that can generate a fully differentiated functional cell of a more than one given cell type. The role of stem cells iii vivo is to replace cells that are destroyed during the normal life of an animal. Generally, stem cells can divide without limit. After division, the stem cell may remain as a stem cell, become a precursor cell, or proceed to terminal differentiation. Although appearing morphologically unspecialized, the stem cell may be considered differentiated where the possibilities for further differentiation are limited. A precursor cell is a cell that can generate a fully differentiated functional cell of at least one given cell type. Generally, precursor cells can divide. After division, a precursor cell can remain a precursor cell, or may proceed to terminal differentiation. A “pancreatic stem cell” is a stem cell of the pancreas. In one embodiment, a pancreatic stem cell gives rise to all of the pancreatic endocrine cells, e.g. the α cells, β cells, δ cells, and PP cells, but does not give rise to other cells such as the pancreatic exocrine cells. A “pancreatic precursor cell” is a precursor cell of the pancreas. In one embodiment, a pancreatic precursor cell gives rise to more than one type of pancreatic endocrine cell. One specific, non-limiting example of a pancreatic precursor cell is a cell that give rise to α and β cells.

Subject refers to any mammal, such as humans, non-human primates, pigs, sheep, cows, rodents and the like which is to be the recipient of the particular treatment. In one embodiment, a subject is a human subject or a murine subject.

Therapeutic agent: Used in a generic sense, it includes treating agents, prophylactic agents, and replacement agents.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses {Wolff, J. A., ed, Gene Therapeutics, Birkhauser, Boston, USA (1994)}. In the case of infection by retroviruses, the infecting retrovirus particles are absorbed by the target cells, resulting in reverse transcription of the retroviral RNA genome and integration of the resulting provirus into the cellular DNA. Methods for the introduction of genes into the pancreatic endocrine cells are known (e.g. see U.S. Pat. No. 6,110,743, herein incorporated by reference). These methods can be used to transduce a pancreatic endocrine cell produced by the methods described herein, or an articficial islet produced by the methods described herein.

Genetic modification of the target cell is an indicium of successful transfection. “Genetically modified cells” refers to cells whose genotypes have been altered as a result of cellular uptakes of exogenous nucleotide sequence by transfection. A reference to a transfected cell or a genetically modified cell includes both the particular cell into which a vector or polynucleotide is introduced and progeny of that cell.

Transgene: An exogenous gene supplied by a vector.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### Method of Producing Pancreatic Endocrine Cells

The methods and cells described herein are based on the discovery that embryonic stem cells can be differentiated in vitro to form any tissue of interest. Thus, pancreatic embryonic stem cells can be differentiated to form endocrine cells. In one embodiment, a method is provided to differentiate embryonic stem cells to pancreatic endocrine cells.

The method includes generating embryoid bodies from a culture of undifferentiated embryonic stem cells, selecting endocrine precursor cells, expanding the endocrine precursor cells by culturing endocrine cells in an expansion medium that comprises a growth factor and differentiating the expanded endocrine precursor cells in a differentiation media to differentiated endocrine cells. An example of this method is outlined below.

Expansion of Undifferentiated Embryonic Stem (ES) Cells

The expansion of ES cells prior to differentiation is not required to perform the method disclosed herein. However, to increase the number of pancreatic endocrine cells formed, ES cells can be expanded prior to embryoid body formation. Undifferentiated embryonic stem (ES) cells are cultured in ES proliferation media to expand the number of cells. Without being bound by theory, it is believed that ES cells can be expanded at least about 1000 fold without losing pluripotency. In one embodiment, the ES cells are mammalian ES cells. In one specific, non-limiting example, the cells are non-human ES cells, for example primate, sheep, cow, pig, rat, or mouse ES cells. In another embodiment, the ES cells are human ES cells such as human ES cells such as H9.1 or H9.1 (Amit et al., Devel. Bio. 227: 271-8, 2000; Thomson et al., Science 282, 5391, 1998) or human embryonic germ cells (EG cells) (Shamblot et al., Proc. Natl. Acad. Sci. USA 95, 13726, 1998). In one specific non-limiting example the cells are murine ES cells such as E14.1 cells, R1 cells, B5 cells (Hadjantonakis et al., Mech. Dev. 76, 79 (1998); Kao et al., Ophthalmol. Vis. Sci. 37, 2572 (1996).

The ES cells are cultured in an ES growth medium which generally includes a carbon source, a nitrogen source and a buffer to maintain pH. In one embodiment, ES growth medium contains a minimal essential medium, such as DMEM, supplemented with various nutrients to enhance ES cell growth. Additionally, the minimal essential medium may be supplemented with additives such as horse, calf or fetal bovine serum (for example, from between about 10% by volume to about 20% by volume or about 15% by volume) and may be supplemental with nonessential amino acids, L-glutamine, and antibiotics such as streptomycin, penicillin, and combinations thereof. In addition, 2-mercaptoethanol may also be included in the media. ES growth media is commercially available, for example as KO-DMEM (Life-Tech Catalog No. 10829-018).

Other methods and media for obtaining and culturing embryonic stem cells are known and are suitable for use (Evans et al., Nature 292:154-156, 1981; Martin et al., Proc. Natl. Acad. Sci. 78:7634-7636, 1981; Robertson et al., Nature 323:445-448, 1986; Doetschman et al., Nature 330:576-578, 1987; “Thomas et al., Cell 51:503-512, 1987; Thomson et al., Science 282:1145-1147, 1998; and Shamblott et al., Proc. Natl. Acad. Sci. U.S.A. 95:13726-13731, 1998). The disclosures of these references are incorporated by reference herein.

In one specific, non-limiting example, the ES cells are cultured on plates which prevent differentiation of the ES cells. Suitable plates include those such as gelatin coated tissue culture plates, or plates which include a feeder cell layer such as a fibroblast feeder cell layer (e.g. mouse embryonic cell line (STO-1) or primary mouse embryonic fibroblasts, both treated with ultra-violet light or an anti-proliferative drug such as mitomycin C). The ES cells are cultured in the presence of LIF (Leukemia Inhibitory Factor), a growth factor that prevents differentiation of ES cells. In one embodiment, the ES cells are cultured for about 4 days to about 8 days. In another embodiment, the ES cells are cultured for about 6 days to about 7 days. The ES cells are cultured at temperature between about 35° C. and about 40° C., or at about 37° C. under an atmosphere which contains oxygen and between from about 1% to about 10%, or from about 1% to 5% CO₂, or at about 5% CO₂. In one embodiment, the media is changed about every 1 to 2 days (see U.S. Pat. No. 5,670,372, herein incorporated by reference).

Generation of Embryoid Bodies

In one embodiment, embryoid bodies are generated in suspension culture. Briefly, to form embryoid bodies, clusters of ES cells are disengaged from the tissue culture plates. Methods for disengaging cells from tissue culture plates are known and include the use of enzymes, such as trypsin or papain, and/or methyl ion chelators such as EDTA or EGTA, or commercially available preparations (e.g. see WO 00/27995).

Generally, the ES cells disengage from the tissue culture plates in clusters (e.g., aggregates of 10 or more ES cells, typically 50 or more cells). The clusters of ES cells are then dissociated to obtain a population of cells which includes a majority of (e.g., between about 50% and about 70%, or between about 75% and about 90%, or between about 80% and about 100%) individual cells. Methods for dissociating clusters of cells are likewise known. One method for dissociating cells includes mechanically separating the cells, for example, by repeatedly aspirating a cell culture with pipette. In one embodiment, the ES cells are in an exponential growth phase at the time of dissociation to avoid spontaneous differentiation that tends to occurs in an overgrown culture.

The dissociated ES cells are then cultured in an ES proliferation medium. However, in contrast to the ES cell proliferation (in which the cells are grown on a tissue culture dish surface), embryoid bodies are generated in suspension. For example, to form embryoid bodies, the cells may be cultured on non-adherent bacterial culture dishes. In one embodiment, the cells are incubated from about 4 days to about 7 days, or up to about 8 days. In one embodiment, the medium is changed every 1 to 2 days (see Martin et al., Proc. Natl. Acad. Sci. 72:1441-1445, 1975; U.S. Pat. No. 5,014,268, herein incorporated by reference).

In another embodiment, embryoid bodies are not generated, but undifferentiated ES cells are plated directly in serum-free media for selection of nestin-positive pancreatic stem cells or pancreatic precursor cells, as described below.

Selection of Pancreatic Endocrine Stein Cells

The cells of the embryoid body are cultured to select for pancreatic endocrine stem cells or pancreatic endocrine cell precursors. In one embodiment, to select for pancreatic endocrine stem cells or precursor cells, the EB cells are plated onto a surface that permits adhesion of pancreatic endocrine stem cells or precursor cells, for example a fibronectin-, laminin-, or vitronectin-coated surface. In another embodiment, embryoid bodies are not generated, but ES cells are directly plated onto the surface.

In addition, the cells are cultured using a medium which selects for pancreatic endocrine stem cells precursor cells. In one embodiment, the medium is a serum-free minimal essential medium, such as DMEM or F12, or a combination of DMEM and F12. The serum-free medium is supplemented with nutrients. Specific, non-limiting examples of nutrients are insulin, selenium chloride, transferrin and fibronectin. An example of a serum free media is ITSFn medium which includes DMEM and F12 in a ratio between 0.1:1 and 10:1 supplemented with between about 1 μg/ml to about 10 μg/ml insulin, about 20 nM to about 40 nM selenium chloride, about 40 μg/nl to about 60 μg/ml transferrin and between about 1 μg/ml to 10 μg/ml fibronectin. In one embodiment, the cells are incubated in the serum-free medium for between about 6 to about 8 days at a temperature between about 35° C. and about 40° C. In another embodiment, the cells are incubated at 37° C. under between about 1% and 10% CO₂ atmosphere, or between about 5% and 10% CO₂ or under about 5% CO₂. In this embodiment, the medium is changed every 1 to 2 days.

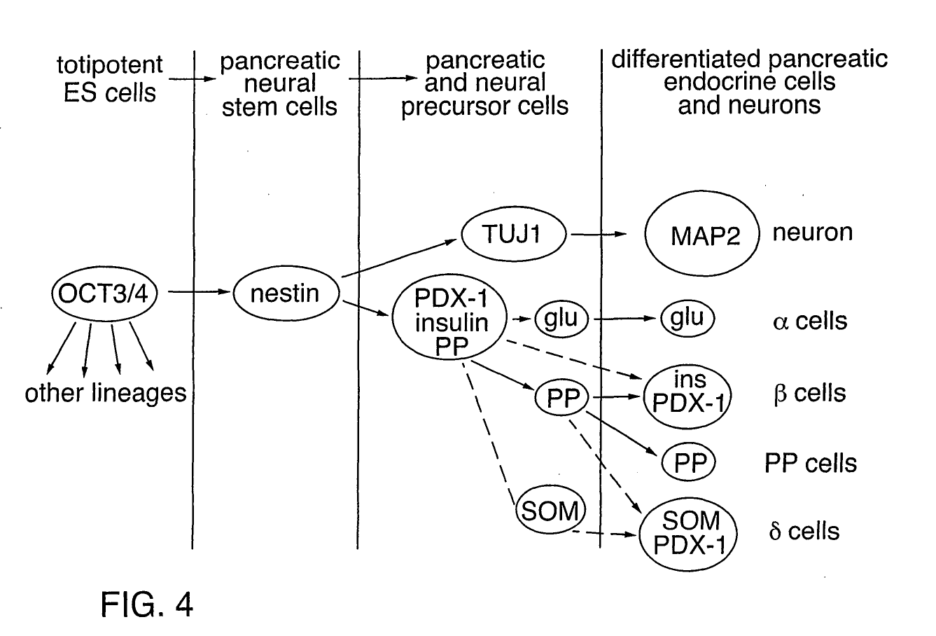

At the end of the selection, the cell culture contains more than about 50% pancreatic endocrine stem cells or precursor cells. In another embodiment, the cell culture contains more than about 80% pancreatic endocrine stem cells or precursor cells, or more than about 90% pancreatic endocrine stem cells or precursor cells. In one embodiment, the pancreatic endocrine stem cells or precursor cells are identified by expression of nestin. Additionally, other polypeptides or transcriptional regulators, typical of the pancreatic endocrine cells, can be identified. One specific, non-limiting example of such a transcriptional regulator is PDX-1. In one embodiment, expression of insulin, glucagon, somatostatin, pancreatic polypeptide is assessed. In other embodiments, NK-X2.2, NKX6.1, IAPP, glut-2, ISL1, neurogenin 3, PAX4, PAX6, neuroD, a member of the LIM homeodomain transcription factor family, is identified (for review see Sender and German, J. Molec. Med. 75:327-40, 1997; Sender et al., Develop. 127:5533-5540, 2000, also see FIG. 4).

Expansion of Pancreatic Stem Cells

In one embodiment, the pancreatic stem cells or precursor cells are expanded until the amount of cells increases about 10 fold. In another embodiment, the pancreatic stem cells or precursor cells are expanded until the amount of cells increases from about 10 fold to about 100 fold. In one specific, non-limiting example, nestin positive cells are expanded in the presence of a growth factor. In another specific, non-limiting example, pancreatic stem cells or precursor are expanded in the presence of a growth factor for about 6 to about 7 days.

A variety of culture media are known and are suitable for use in this step. Generally, the proliferation medium includes a minimal essential medium. In one embodiment, the medium is DMEM and/or F12, or a combination of DMEM and F12 (at a ratio between about 0.1:1 to about 10:1). In another embodiment, the culture medium includes N2 medium.

In one embodiment, the minimal essential medium is supplemented with B27 media supplement (Gibco BRL, Gaithersburg, Mass.) and nicotinamide (Sigma, St. Louis, Mo.). In one embodiment, B27 is provided as a 50× concentrate. B27 is then diluted in the minimal essential media from about 0.5× to about 2× final concentration. In another embodiment, B27 is added to a 1× final concentration in the minimal essential medium. B27 is a supplement that has been shown to have effects on neuron survival in vitro (Brewer et al., J. Neurosci. Res. 35:567, 1993, herein incorporated by reference).

In one embodiment, nicotinamide is added to the minimal essential medium. In one specific, non-limiting example, nicotinamide is added at a concentration of about 1 mM to about 50 mM. In another specific, non-limiting example, nicotinamide is added at concentration of at least about 5 mM and at most about 50 mM. In a further embodiment, nicotinamide is added at a concentration of about 5 mM to about 10 mM. In yet another specific, non-limiting example, nicotinamide is added at a concentration of about 10 mM.

In one embodiment, the medium contains one or more additional additives such as nutrients. Specific, non-limiting examples of these nutrients are shown in the table below

| Additive | Exemplary Concentration |
|---|---|
| glucose | about 0.5 mg/ml to about 5.0 mg/ml |
| glutamine | about 0.01 mg/ml to about 0.1 mg/ml |
| sodium bicarbonate (NaHCO₃) | about 0.05 mg/ml to about 5.0 mg/ml |
| insulin | about 10 mg/ml to about 30 mg/ml |
| transferrin | about 50 mg/ml to about 150 mg/ml |
| putrescine | about 50 μM to about 150 μM |
| selenite | about 20 nM to about 40 nM |
| progesterone | about 10 nM to about 30 nM |

Thus, in one embodiment, the medium includes between about 0.05 mg/ml and about 5.0 mg/ml sodium bicarbonate. In another embodiment, the medium includes between about 1.0 mg/ml to about 2.0 mg/ml sodium bicarbonate. In another embodiment the medium does not include 4-(2-hydroxyethyl)-1-piperazine-ethanesulfonic acid (HEPES).

The pancreatic stem cell proliferation media can also be supplemented with growth factors. In one specific, non-limiting example, the proliferation medium includes basic fibroblast growth factor (bFGF). In one embodiment, the culture medium includes between about 5 ng/ml to about 30 ng/ml of bFGF. In another embodiment, the medium includes about 10 ng/ml to about 20 ng/ml bFGF. In yet another embodiment, the proliferation medium includes between about 10 ng/ml and about 20 ng/ml bFGF.

In another specific, non-limiting example, the proliferation medium includes epidermal growth factor (EGF). In one embodiment, the culture medium includes between about 5 ng/ml to about 30 ng/ml of EGF. In another embodiment, the medium includes about 10 ng/ml to about 20 ng/ml EGF. In yet another embodiment, the proliferation medium includes between about 10 ng/ml and about 20 ng/ml EGF. The culture medium may also be supplemented with additional agents to increase the efficiency of the generation of pancreatic endocrine cells.

In yet another embodiment, other biological active molecules or growth factors are added. Growth factors include, but are limited to, cilliary neurotrophic growth factor (CNGF, Gupta S K et al. J Neurobio. 23: 481-90, 1992), a neurotrophin such as neurotrophin-3, neurotrophin-4, nerve growth factor (NCF) (Kaplan and Miller, Curr. Opin. Neurobiol. 10:381-391, 2000), and glial derived neurotrophic factor (GDNF), hepatocyte growth factor (HGF), beta-cellulin, activin A, activin B, bone morphogenic proteins (BMP-2, BMP-4), transforming growth factor β (TGF-β), noggin (see Itoh et al., Eur. J. Biochem. 267:6954-6967, 2000). Biologically active agents include, but are not limited to ascorbic acid, cyclic AMP (cAMP) and retinoic acid (e.g. trans-retinoic acid).

In a further specific, non-limiting example the proliferation media includes erythropoietin (EPO). For example the media can include from about 10 ng/ml to about 50 ng/ml, or from about 0.1 U/ml to about 5 U/ml, or from about 0.5 U/ml to about 5 U/ml (Studer et al., J. Neurosci. 20:7377-7383, 2000).

In one embodiment, the cells are cultured under conditions under an oxygen concentration of about 20% (atmospheric oxygen). In another embodiment, the cells are cultured under conditions of low atmospheric oxygen concentration (Studer et al., J. Neurosci. 20:7377-7383, 2000). Specific, non-limiting of low atmospheric oxygen concentration are from about 1% oxygen to about 5% oxygen. In another specific, non-limiting example, the cells are cultured from about 1% to about 20% oxygen. In another embodiment, the cells are incubated at about 37° C. under between about 1% and 10% CO₂, or between about 5% and 10% CO₂ or at about 5% CO₂. In a specific, non-limiting example, the medium is changed every 1 to 2 days.

Differentiation of the Expanded Pancreatic Endocrine Cell Stem Cells or Precursors

Differentiation of the expanded pancreatic endocrine cell stem cells or pancreatic endocrine precursor cells to form mature endocrine cells is induced by withdrawal of at least one growth factor such as bFGF (or EGF) (see above exapansion of pancreatic endocrine cell stem cells). In one embodiment, differentiation is induced by culturing the cells in medium similar to the culture medium, but without at least one agent (e.g., bFGF or EGF). In one embodiment, the medium includes B27 supplement and nicotinamide. Additionally, the medium may contain factors to enhance the yield of pancreatic endocrine cells. In one embodiment, the expanded cell population still expresses nestin.

In yet another embodiment, other biological active molecules are included in the media. These factors can include, but are limited to, cilliary neurotrophic growth factor (CNGF, Gupta SK et al. J. Neurobio. 23: 481-90, 1992), a neurotrophin such as neurotrophin-3, neurotrophin-4, nerve growth factor (NCF) (Kaplan and Miller, Curr. Opin. Neurobiol. 10:381-391, 2000), and glial derived neurotrophic factor (GDNF), hepatocyte growth factor (HGF), beta-cellulin, activin A, activin B, bone morphogenic proteins (BMP-2, BMP-4), transforming growth factor β (TGF-β), noggin (see Itoh et al., Eur. J. Biochem. 267:6954-6967, 2000). Biologically active agents include, but are not limited to ascorbic acid, cyclic AMP (cAMP) and retinoic acid (e.g. trans-retinoic acid).

In a further specific, non-limiting example the proliferation media includes erythropoietin (EPO). For example the media can include from about 10 ng/ml to about 50 ng/ml, or from about 0.1 U/ml to about 5 U/ml, or from about 0.5 U/ml to about 5 U/ml (Studer et al., J. Neurosci. 20:7377-7383, 2000).

In one embodiment, the cells are cultured under conditions under an oxygen concentration of about 20% (atmospheric oxygen). In another embodiment, the cells are cultured under conditions of low atmospheric oxygen concentration (Studer et al., J. Neurosci. 20:7377-7383, 2000). Specific, non-limiting of low atmospheric oxygen concentration are from about 1% oxygen to about 5% oxygen. In another specific, non-limiting example, the cells are cultured from about 1% to about 20% oxygen. In another embodiment, the cells are incubated at about 37° C. under between about 1% and 10% CO₂, or between about 5% and 10% CO₂ or at about 5% CO₂. In a specific, non-limiting example, the medium is changed every 1 to 2 days

The differentiation of pancreatic stem cells into pancreatic endocrine cells can be measured by any means known to one of skill in the art. Specific, non-limiting examples are immunohistochemical analysis to detect a pancreatic endocrine polypeptides (e.g. insulin, glucagon, somatostatin, or pancreatic polypeptide), or assays that detect the secretion of the pancreatic endocrine polypeptides (e.g. see U.S. Pat. No. 5,993,799; Csernus et al., Cell. Mol. Life Sci. 54, 733,1998; Alpert, Cell 53:295-308, 1988), or assay such as ELISA assays and Western blot analysis. Differentiation of cells can also be measured by measuring the level of mRNA coding for pancreatic endocrine polypeptides such as Northern blot, RNase protection and RT-PCR (Clark et al., Diabetes 46:958-967, 1997; Hebrok et al., Genes and Dev. 12: 1705-1713, 1998).

### Method of Producing Artificial Islets

In one embodiment pancreatic endocrine cells are produced as described above and artificial islets are generated. In one embodiment, the artificial islet is produced by culturing methods as described above. In this embodiment, the pancreatic endocrine cells, generated as described above are used directly. In another embodiment pancreatic endocrine cells are dislodged. In another embodiment, pancreatic endocrine cells produced in vitro and disassociated, a cell suspension is made, and the cells are then re-aggregated.

An artificial pancreatic islet includes at least one type of pancreatic endocrine cell. In one embodiment, the artificial islet includes pancreatic β cells. In another embodiment, the artificial islet includes the α cells. In yet another embodiment, the artificial islet includes the δ cells. In a further embodiment, the artificial islet includes more than one pancreatic endocrine cell type. In a specific, non-limiting example, an artificial islet includes the pancreatic β cells in addition to another pancreatic endocrine cell type, such as, but not limited to, the pancreatic α cells. In a specific, non-limiting example, an artificial islet includes the pancreatic β cells in addition to another pancreatic endocrine cell type, such as, but not limited to, the pancreatic δ cells.

A pancreatic endocrine cell produced by the methods described herein, or an artificial islet produced by the methods described herein can be transduced or transfected with a nucleic acid sequence of interest. Transfection refers to the introduction of an exogenous nucleotide sequence, such as DNA vectors in the case of mammalian target cells, into a target cell, whether or not any coding sequences are ultimately expressed.

### Use of Pancreatic Endocrine Cell Produced to Study Agents that Affect Islets and/or the Secretion of Pancreatic Endocrine Hormones

Another aspect of the invention provides an assay for evaluating the effect of substances on pancreatic endocrine cells. The assay can be used to test agents capable of regulating the survival, proliferation, or genesis of pancreatic endocrine cells. According to this aspect of the invention, a population of pancreatic endocrine cells or their precursors is produced as described above. The population of cells is contacted with a substance of interest and the effect on the cell population is then assayed.

In one specific, non-limiting example, pancreatic endocrine cells differentiated from embryonic stem cells are contacted with an agent of interest. A parameter is then assayed to determine if the agent affects the pancreatic endocrine cells. In one specific non-limiting example, the secretion or expression of a pancreatic endocrine hormone is analyzed. Specifically, the secretion or expression of insulin, glucagon, somatostatin, or pancreatic polypeptide can be analyzed. Alternatively, if the pancreatic endocrine cells are transfected with a nucleic acid construct encoding a reporter gene an increase or decrease in the expression of the reporter gene can be analyzed (see bleow). This analyses can include detection of the level of protein or RNA present in the pancreatic endocrine cell, or can include detection of the biological activity of the reporter gene.

Substances of interest include extracts from tissues or cells, conditioned media from primary cells or cell lines, polypeptides whether naturally occurring or recombinant, nucleotides (DNA or RNA) and non-protein molecules whether naturally occurring or chemically synthesized.

Pancreatic endocrine cells differentiated from embyronic stem cells can also be used to as a model system to study the biology of the pancreatic islets. Specific, non-limiting examples are in vitro studies of insulin secretion, proliferation of the pancreatic endocrine cells, and malignant transformation of the pancreatic endocrine cells.

Pancreatic endocrine cells differentiated from ES cells can also be used to evaluate the role of various genes in differentiated pancreatic endocrine cells. For example, a specific gene may be “knocked out” in an ES cell. A gene knock-out is the targeted disruption of a gene in vivo with complete loss of function that has been achieved by any transgenic technology familiar to those in the art. In one embodiment, animals having gene knockouts are those in which the target gene has been rendered nonfunctional by an insertion targeted to the gene to be rendered non-functional by homologous recombination. Methods for producing knock out variants are known (e.g. see Shastry, Mol. Cell Biochem. 181:163-179, 1998). The ES cell including a knocked out gene (for example, a homozygous null mutant) can be cultured to form differentiated pancreatic endocrine cells deficient for the gene product.

In another embodiment, transgenic animals can be produced by introducing into embryos (e.g. a single celled embryo) a polynucleotide, in a manner such that the polynucleotide is stably integrated into the DNA of germ line cells of the mature animal and inherited in normal Mendelian fashion. Advances in technologies for embryo micromanipulation now permit introduction of heterologous DNA into fertilized mammalian ova. For instance, totipotent or pluripotent stem cells can be transformed by microinjection, calcium phosphate mediated precipitation, liposome fusion, viral infection or other means, the transfected cells are then introduced into the embryo, and the embryo then develops into a transgenic animal.

In one method DNA is injected into the pronucleus or cytoplasm of embryos, preferably at the single cell stage, and the embryos allowed to develop into mature transgenic animals. These techniques are well known. For instance, reviews of standard laboratory procedures for microinjection of heterologous DNAs into mammalian (mouse, pig, rabbit, sheep, goat, cow) fertilized ova include: Hogan et al., Manipulating the Mouse Embryo, Cold Spring Harbor Press, 1986; Krimpenfort et al., Bio/Technology 9:86, 1991; Palmiter et al., Cell 41:343, 1985; Kraemer et al., Genetic Manipulation of the Early Mammalian Embryo, Cold Spring Harbor Laboratory Press, 1985; Hammer et al., Nature, 315:680, 1985; Purcel et al., Science, 244:1281, 1986; Wagner et al., U.S. Pat. No. 5,175,385; Krimpenfort et al., U.S. Pat. No. 5,175,384, the respective contents of which are incorporated by reference. The transgenic mice can then be used to generate ES cells including a transgene, which can be differentiated into pancreatic endocrine cells by the methods described herein.

In another embodiment, nuclear transfer technologies can be used to derive autologous human ES cells (Coleman and Kind, Trends Biotechnol. 18:192-196, 2000). These cells are then used to differentiate pancreatic islet cells that will be rejected by the immune system. In another example, other stem cells, such as bone marrow stem cells are de-differentiated into pluripotent stem cells, and these pluripotent stem cells are subsequently differentiated to cells of the pancreatic lineage (Jackson et al., Proc. Natl. Acad. Sci. USA 96:14482-14486, 1999).

### Transfection of Pancreatic Endocrine Cells Differentiated from Embryonic Stem Cells

In an additional embodiment of the invention, ES cell or pancreatic endocrine cells differentiated from an ES cell may be transfected with a heterologous nucleic acid sequence. In one embodiment, the heterologous nucleic acid sequence encodes polypeptide of interest. In one embodiment, the polypeptide of interest encodes any polypeptide or protein that is involved in the growth, development, metabolism, enzymatic or secretory pathways in a pancreatic endocrine cell. Such polypeptides may be naturally occurring pancreatic hormones, proteins, or enzymes, or may be fragments thereof. In another embodiment, the polypeptide encodes a marker. In yet another embodiment, the polypeptide is an enzyme involved in the conversion of a pro-drug to an active agent.

According to this aspect of the invention, cells are cultured in vitro as described herein and an exogenous gene encoding the heterologous nucleic acid is introduced into the cells, for example, by transfection. The transfected cultured cells can then be studied in vitro or can be administered to a subject (see below).

The polypeptide encoded by the nucleic acid can be from the same species as the cells (homologous), or can be from a different species (heterologous). For example, a nucleic acid sequence can be utilized that supplements or replaces deficient production of a peptide by the tissue of the host wherein such deficiency is a cause of the symptoms of a particular disorder. In this case, the cells act as a source of the peptide. In one specific, non-limiting example the polypeptide is insulin. Thus, in one specific, non-limiting example, a nucleic acid sequence encoding human insulin is introduced into a human cell. In another specific, non-limiting example, a nucleic acid encoding human insulin is introduced into a murine cell.

In one embodiment, the nucleic acid of interest encodes a polypeptide involved in growth regulation or neoplastic transformation of endocrine cells. Specific, non-limiting examples of nucleic acids sequences of interest are SV40 Tag, p53, myc, src, and bcl-2. In another embodiment, the nucleic acid sequence of interest encodes an enzyme. Specific, non-limiting examples of enzymes are proteins involved in the conversion of a pro-drug to a drug, or enzymes involved in the conversion of preproinsulin to proinsulin, or proinsulin to insulin, or growth factors that promote the expansion, differentiation, or survival of pancreatic progenitor cells, such as neurotrophins, bFGF, activin A, and activin B. In yet another embodiment, the nucleic acid sequence of interest encodes a transcriptional regulator. Specific, non-limiting examples of a transcriptional regulator are PDX-1, PAX4, neurogenin3, and NKX2.2. Without being bound by theory, introduction of nucleic acid sequences encoding transcriptional regulators can permit more efficient commitment of a early progenitor cell to the pancreatic endocrine lineage. Introduction of a nucleic acid sequence encoding a transcriptional regulator can also permit more efficient proliferation and differentiation of the committed pancreatic progenitor. In addition, introduction of a nucleic acid encoding transcriptional regulators can increase survival of a pancreatic progenitor cell during in vitro culture and/or after transplantation of the cell in vivo.

In yet another specific, non-limiting example, a nucleic acid sequence can be introduced to decrease rejection. For example, the immunogenicity of a cell may be suppressed by deleting genes that produce proteins that are recognized as “foreign” by the host (a knock-out), or by introducing genes which produce proteins, such as proteins that are native to the host and recognized as “self” proteins by the host immune system.

In one embodiment, the nucleic acid sequence of interest is operably linked to a regulatory element, such as a transcriptional and/or translational regulatory element. Regulatory elements include elements such as a promoter, an initiation codon, a stop codon, mRNA stability regulatory elements, and a polyadenylation signal. A promoter can be a constitutive promoter or an inducible promoter. Specific non-limiting examples of promoters include the CMV promoter, an insulin promoter, and promoters including TET-responsive element for inducible expression of transgene. In another embodiment, the nucleic acid sequence of interest and inserted into a vector, such as an expression vector. Procedures for preparing expression vectors are known to those of skill in the art and can be found in Sambrook et al., Molecular Cloning: A Laboratory Manual, 2nd Ed., Cold Spring Harbor Press, Cold Spring Harbor, N.Y. (1989). Expression of the nucleic acid of interest occurs when the expression vector is introduced into an appropriate host cell.

In another embodiment, an ES may be transfected with a nucleic acid designed to functionally delete or “knock-out” a gene of interest. In this method, the nucleic acid of interest is a nucleic acid that undergoes homologous recombination and is inserted into the genome of the ES cell. Methods for producing “knock-outs” in ES cells are known to one of skill in the art (e.g. see U.S. Pat. No. 5,939,598, herein incorporated by reference).

In one embodiment, the host cell for transfection is an ES cell (Levinson-Dushnik and Benvenifty, Mol. Cell. Biol. 17:3817-3822, 1997). Thus, upon differentiation, the ES cell transfected with the nucleic acid sequence of interest generates a pancreatic stem cell or precursor cell including the nucleic acid sequence of interest. The pancreatic stem cell or precursor cell can then be differentiated into a pancreatic endocrine cell including the nucleic acid sequence of interest.

In another embodiment, the host cell is a pancreatic endocrine stem cell or precursor cells. Upon differentiation, the pancreatic endocrine stem cell or precursor cells can differentiate into a pancreatic endocrine cell including the nucleic acid sequence of interest. In yet another embodiment, the host cell is a pancreatic endocrine cell differentiated from an ES cell such as a pancreatic endocrine cell in an artificial islet. Methods for the introduction of nucleic acid sequences into pancreatic endocrine cells or into embryonic stem cells are known in the art (e.g., see U.S. Pat. No. 6,110,743, herein incorporated by reference).

### Transplantion of Pancreatic Endocrine Cells Differentiated from ES Cells

In another embodiment, the invention provides a method of treating a subject suffering from a disease or disorder, such as a endocrine system disorder, or alleviating the symptoms of such a disorder, by administering cells cultured according to the method of the invention to the subject. Examples of endocrine disorders included disorders of the pancreatic endocrine system, such as type I or type II diabetes.

In one embodiment, cells are cultured as described herein to form differentiated pancreatic endocrine cells or artificial islets. The pancreatic endocrine cells or artificial islets are then administered to the subject.

Formulations

After the differentiated pancreatic endocrine cells are differentiated according to the cell culturing method previously described, the cells or artificial islets are suspended in a pharmacologically acceptable carrier. Specific, non-limiting examples of suitable carriers include cell culture medium (e.g., Eagle's minimal essential media), phosphate buffered saline, Krebs-Ringer buffer, and Hank's balanced salt solution +/− glucose (HBSS).

The volume of cell suspension administered to a subject will vary depending on a number of parameters including the size of the subject, the severity of the disease or disorder, and the site of implantation and amount of cells in solution. Typically the amount of cells administered to a subject will be a therapeutically effective amount.

It is estimated that a diabetic subject will need at least about 1, 000, or between 1,000 and 10,000, or between 1,000 and 100,000 surviving insulin producing cells per transplantation to have a substantial beneficial effect from the transplantation.

Methods of Administration

The pancreatic endocrine cells differentiated from embryonic stem cells can be administered by any method known to one of skill in the art. In one specific, non-limiting example the cells are administered by sub-cutaneous injection, or by implantation under the kidney capsule, through the portal vein of the liver, or into the spleen. In one embodiment, about 1,000 to about 10,000 cells are implanted. If, based on the method of adminsitration, cell survival after transplantation in general is low (5-10%) an estimated 1-4 million pancreatic endocrine cells are transplanted.

In one embodiment, a transplantation is made by injection. Injections can generally be made with a sterilized syringe having an 18-23 gauge needle. Although the exact size needle will depend on the species being treated, and whether a cell suspension or an artificial islets is transplanted, the needle should not be bigger than 1 mm diameter in any species. The injection can be made via any means known to one of skill in the art. Specific, non-limiting examples include subcutaneous injection, intra-peritoneal injection, injection under the kidney capsule, injection through the portal vein, and injection into the spleen.

In one embodiment, the cells are directly administered to a subject. In another embodiment, the cells are encapsulated prior to administration, such as by co-incubation with a biocompatible matrix known in the art. A variety of encapsulation technologies have been developed (e.g. Lacy et al., Science 254:1782-84, 1991; Sullivan et al., Science 252:7180712, 1991; WO 91/10470; WO 91/10425; U.S. Pat. No. 5,837,234; U.S. Pat. No. 5,011,472; U.S. Pat. No. 4,892,538, each herein incorporated by reference).

Pancreatic endocrine cells may be implanted using an alginate-polylysine encapsulation technique (O'Shea and Sun, Diabetes 35:943-946, 1986; Frischy et al. Diabetes 40:37, 1991). In this method, the cells are suspended in 1.3% sodium alginate and encapsulated by extrusion of drops of the cell/alginate suspension through a syringe into CaCl₂. After several washing steps, the droplets are suspended in polylysine and rewashed. The alginate within the capsules is then reliquified by suspension in 1 mM EGTA and then rewashed with Krebs balanced salt buffer. Each capsule is designed to contain several hundred cells and have a diameter of approximately 1 mm. Capsules containing cells are implanted (approximately 1,000-10,000/animal) intraperitoneally and blood samples taken daily for monitoring of blood glucose and insulin.

Other methods for implanting islet tissue into mammals have been described (Lacy et al., supra, 1991; Sullivan et al., supra, 1991; U.S. Pat. No. 5,993,799, each incorporated herein by reference). In one specific, non-limiting example, islets are encapsulated in hollow acrylic fibers and immobilized in alginate hydrogel. These fibers are then transplanted intraperitoneally or subcutaneously implants.

In another embodiment, pancreatic endocrine cells derived from embryonic stem cells can be administered as part of a biohybrid perfused “artificial pancreas”, which encapsulates islet tissue in a selectively permeable membrane (Sullivan et al., Science 252: 718-721, 1991). In this method, a tubular semi-permeable membrane is coiled inside a protective housing to provide a compartment for the islet cells. Each end of the membrane is then connected to an arterial polytetrafluoroethylene (PTFE) graft that extends beyond the housing and the device is joined to the vascular system as an arteriovenous shunt. Other suitable methods are known to those of skill in the art.

Without further elaboration, it is believed that one skilled in the art can, using this description, utilize the present invention to its fullest extent. The following examples are illustrative only, and not limiting of the remainder of the disclosure in any way whatsoever.

### EXAMPLES

### Example 1

### Method of Generating Pancreatic Endocrine Cells

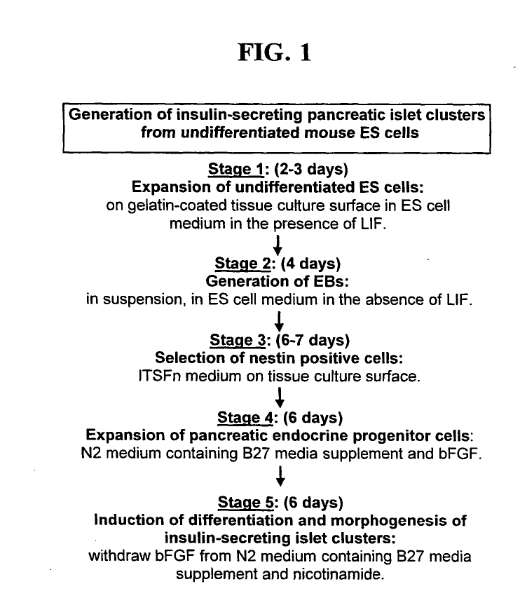

The experimental strategy is outlined in FIG. 1A. A population of nestin-positive cells was generated from embryoid bodies (EBs, stage 2) by selection in serum-free medium (stage 3). Nestin-positive cells were then expanded in the presence of a mitogen, basic fibroblast growth factor (bFGF, stage 4), followed by differentiation of nestin-positive progenitors after mitogen withdrawal (stage 5).

To improve the yield of pancreatic endocrine cells, the culture system was modified by including B27 media supplement (Brewer et al., J. Neurosci. Res. 35:567, 1993), and nicotinamide (Otonkoski et al., J Clin. Invest. 92:1459, 1993) as outlined in FIG. 1A. Specifically, B27 media supplement (Gibco BRL, Gaithersburg, Mass.) was added at concentration recommended by the manufacturer; nicotinamide (Sigma, St. Louis, Mo.) was added at concentration 10 mM. A RT/PCR analysis was then performed on nucleic acid extracted from the cells.

Total cellular RNA purification and RT/PCR was carried out as previously described (Lee et al., Nat. Biotechnol. 18:675, 2000). Identity of the PCR products was confirmed by sequencing. Forward and reverse primer sequences from 5′ to 3′ direction and the length of the amplified products were as follows:

| | | |
|---|---|---|
| insulin I: | TAGTGACCAGCTATAATCAGAG; | (SEQ ID NO: 1) |
| | ACGCCAAGGTCTGAAGGTCC; | (SEQ ID NO: 2)-288bp |
| insulin II: | CCCTGCTGGCCCTGCTCTT; | (SEQ ID NO: 3) |
| | AGGTCTGAAGGTCACCTGCT; | (SEQ ID NO: 4)-212bp |
| glucagon: | TCATGACGTTTGGCAAGTT; | (SEQ ID NO: 5) |
| | CAGAGGAGAACCCCAGATCA; | (SEQ ID NO: 6)-202bp |
| IAPP: | GATTCCCTATTTGGATCCCC; | (SEQ ID NO: 7) |
| | CTCTCTGTGGCACTGAACCA; | (SEQ ID NO: 8)-221bp |
| Glut2: | AGCTTTTCTTTGCCCTGAC; | (SEQ ID NO: 9) |
| | CCCTGGGATGAAGAGGAGAC; | (SEQ ID NO: 10)-541bp |
| PDX-1: | TGTAGGCAGTACGGGTCCTC; | (SEQ ID NO: 11) |
| | CCACCCCAGTTTACAAGCTC; | (SEQ ID NO: 12)-325bp |
| α-amylase-2A: | CATTGTTGCACCTTGTCACC; | (SEQ ID NO: 13) |
| | TTCTGCTGCTTTCCCTCATT; | (SEQ ID NO: 14)-300bp |
| carboxypeptidase A: | GCAAATGTGTGTTTGATGCC; | (SEQ ID NO: 15) |
| | ATGACCAAACTCTTGGACCG; | (SEQ ID NO: 16)-521bp |
| β-actin: | ATGGATGACGATATCGCTG; | (SEQ ID NO: 17) |
| | ATGAGGTAGTCTGTCAGGT; | (SEQ ID NO: 18)-568bp |

RT/PCR analysis of endocrine pancreatic gene expression at stage 1 and 5 (FIG. 1B) showed that both forms of murine insulin, insulin I and insulin II (Wentworth et al., J. Mol. Evol. 23:305, 1986) and glucagon (Rothenberg et al., J. Biol. Chem., 270:10136 1995) were expressed at stage 5. Islet amyloid polypeptide (IAPP, Ekawa et al., Mol. Endocrinol. 19:79, 1997) and β cell-specific glucose transporter (Glut2, Waeber et al., J. Biol. Chem. 28:26912, 1994) were also induced. Pancreatic transcription factor PDX-1, known to play an important role in pancreatic development (Ohlsson et al., EMBO J. 12:4251, 1993; Guz et al., Development 121:11, 1995), was expressed in the undifferentiated ES cells. The results of RT/PCR analysis suggest that the differentiation conditions developed support the differentiation of pancreatic cells.

### Example 2

### Identification of Pancreatic Endocrine Cells

Immunocytochemistry was used to identify nestin-positive progenitors, neurons, and insulin-positive cells in the ES cell cultures. Specifically, cells were fixed in 4% paraformaldehyde/0.15% picric acid in PBS. Immunocytochemistry was carried out utilizing standard protocols. The following primary antibodies were used at following dilutions: nestin rabbit polyclonal 1:500 (made in our laboratory), TUJ1 mouse monoclonal 1:500, TUJ2 rabbit polyclonal 1:2000 (both from Babco, Richmond, Calif.), insulin mouse monoclonal 1:1000 (Sigma, St. Louis, Mo.), insulin guinea pig polyclonal 1:100 (DAKO, Carpinteria, Calif.), glucagon rabbit polyclonal 1:75 (DAKO), somatostatin rabbit polyclonal 1:100 (DiaSorin. Stillwater, Minn.), GFP 1:750 polyclonal (Molecular Probes, Eugine, Oreg., BRDU rat monoclonal 1:100 (Accurate, antibodies, Westbury, N.Y.). For detection of primary antibodies fluorescently labeled secondary antibodies (Jackson Immunoresearch Laboratories, West Grove, Pa. and Molecular Probes) were utilized according to methods recommended by the manufacturers.

The intensity of nestin-specific staining increased toward the end of stage 3. Although no insulin-positive cells were detected at stage 1 and 2 (see FIG. 1A), a few insulin-positive cells appeared by the end of stage 3. At the end of stage 4, in the presence of bFGF, many insulin- and TUJ1-positive (neuron-specific β-III tubulin, 31) cells were present. Insulin staining continued to increase after mitogen withdrawal resulting in many intensely stained insulin-positive cells by the end of stage 5. The number and the state of maturation of neurons also increased during this time, and by the end of stage 5 the majority of insulin-positive cells were localized in tight clusters in close association with neurons.

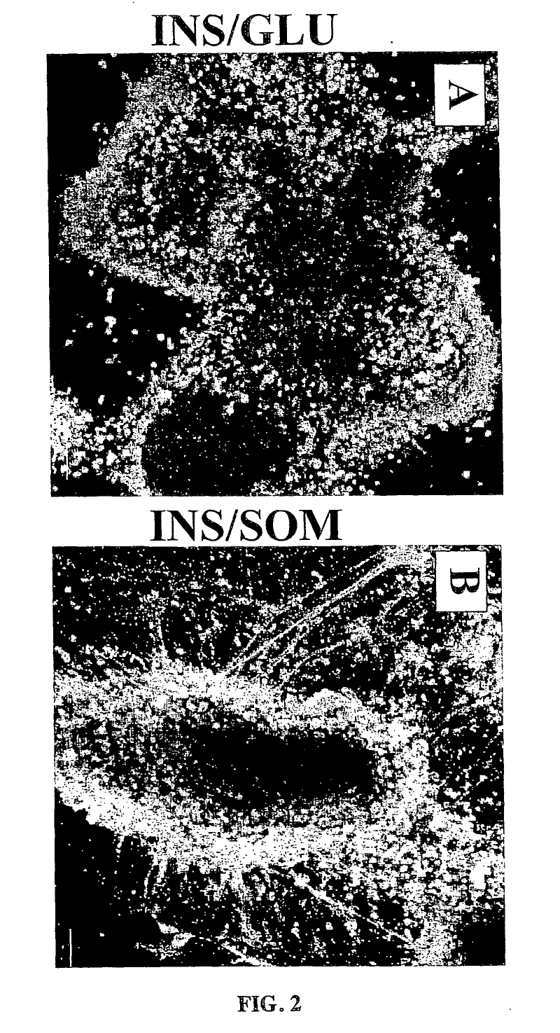

Confocal microscopy was used to analyze the morphology of the cell clusters. A low power image shows that many of the cells in the center of the clusters were insulin-positive (FIG. 2A), and that the neurons grew around and over the insulin-positive cells. This relative special distribution of insulin cells and neurons was particularly apparent in the side view of the cluster. Confocal images failed to detect any TUJ1/insulin double-labeled cells at any developmental stage.

To characterize the differentiation further double immunostaining for insulin and three other pancreatic endocrine hormones was performed: glucagon, somatostatin and pancreatic polypeptide are normally produced by distinct cells in the islets. All three hormones were generated by the cells in the clusters (e.g. FIG. 2). The majority of glucagon and somatostatin cells surround insulin cells. It is important to note that expression of exocrine pancreatic markers amylase and carboxypeptidaseA was not detected by RT/PCR, nor was the expression of amylase detected by immunocytochemistry. The relative distribution of neurons and endocrine cells in this system demonstrates a remarkable capacity of this system to generate multi-cellular structures morphologically analogous to in vivo pancreatic islets.

### Example 3

### Pancreatic Endocrine Cells Generated In Vitro: a Model System to Study the Cells of the Pancreatic Islets

The results described above demonstrate that this ES cell-derived differentiation system provides a powerful tool to investigate the ontogeny and properties of pancreatic progenitors. The analytical capacity of this system was assessed by asking the following questions: (i) is there a common progenitor for pancreatic and neuronal cells in the nestin-positive cell population, (ii) do insulin-positive cells divide, and (iii) at what stage of culture do insulin-negative progenitors initiate insulin expression.

The first question was assessed using clonal analysis. Stage 3 B5 ES cells derived from GFP transgenic mice were co-cultured at clonal density on Poly-Ornitline plus Fibronectin treated-96 well plates (Costar 3603: black plate with clear and thin bottom) with stage 3 E14.5 ES cells at a final concentration of 1 B5 cell/40,000 wild type E14.5 cell/well. Cells were then expanded and differentiated as shown in FIG. 1A. On day 6 of differentiation cells were fixed with 4% paraformaldehyde followed by triple immunocytochemistry and laser confocal analysis. For immunocytochemistry, after the cultures were blocked with 10% normal goat serum/0.3% triton-X100, cells were stained with antibodies against insulin (mouse IgG1), GFP (mouse IgG2a), and TUJ1 (rabbit). Cy5, FITC, and Cy3-conjugated goat antibodies to IgG1 mouse, IgG2a mouse and IgG rabbit respectively were used as secondary antibodies. Clonal cell progeny derived from a single cell were identified by the expression of GFP. GFP labeled clones derived from a single cell were identified in 18-20% of the wells of 96 well plate. Only one GFP labeled clone was present per well.

Specifically, B5 ES cells tagged with green fluorescent protein (GFP, Hadjantonakis et al., Mech. Dev. 76:79, 1998) and wild type E14.1 ES cells (Kao et al., Ophthalmol. Vis. Sci., 37:2572, 1996) were cultured individually through stages 1 to 3 to generate nestin-positive populations. This was followed by co-culture of the two ES cell lines during stages 4 and 5 to obtain individual clones of GFP-labeled B5 cells arising among unlabeled E14.1 cells. Insulin-positive cells were found to express GFP around the area where insulin is localized, and GFP expression was often down-regulated in differentiated cells. Analysis of GFP-positive clones at stage 5 shows that the majority of them contain either neurons or insulin-positive cells. However, rare clones containing both insulin- and TUJ1-labeled cells were seen, suggesting that a common progenitor to neurons and endocrine cells exists in the cell population in the beginning of stage 4 at the time of co-culture initiation.

To answer the second question, the proliferating cells were labeled with bromodeoxyuridine (BrdU) at different time points during the culture followed by immediate cell fixation and immunostaining with antibodies against insulin and BrdU. The cells were labeled with BrdU (Boehringer Mannheim, Indianapolis, Ind.) at final concentration 10 μm for 24 hours. Following the labeling, depending on the specific experiment, the cells were either fixed immediately in 4% paraformaldehyde/0.15% picric acid, treated with 95% ethanol/5% glacial acidic acid for 15 min at room temperature, and subjected to immunocytochemistry, or were cultured for various lengths of time, and then analyzed by immunocytochemistry. The peak of cell proliferation was found to coincide with the end of stage 4, BrdU/insulin double-labeled cells were not detected at any stage. These results suggest that in this ES cell system, similarly to in vitro cultures of normal pancreatic precursors (Vinik et al., Horm. Metab. Res. 29:278, 1997), initiation of insulin expression coincides with inhibition of precursor cell proliferation.

The third question was addressed using BrdU pulse/chase protocol where cells were first labeled with BrdU and then incubated in the absence of BrdU for different periods of time; this step was followed by immunostaining for insulin and BrdU. Quantitative analysis of this experiment defines the switch from proliferation to differentiation. In these studies 8.8+/−2.7% (n=3) of cells proliferating on day 2 of stage 4 had become insulin-positive by day 6 of stage 4. In contrast, 42.2+/−5.9% (n=3) of cells proliferating on day 5 of stage 4 were insulin-positive by day 3 of stage 5. These results establish that significant expansion of pancreatic progenitors takes place at the end of stage 4, and a dramatic shift from proliferation to differentiation occurs at the transition between stages 4 and 5.

### Example 4

### Pancreatic Endocrine Cells In Vitro: a Tool to Study Kinetics and Pharmacology of Insulin Release and to Study Agents that Affect Insulin Secretion

A series of experiments were conducted to measure the kinetics and pharmacology of glucose-dependent insulin release. Insulin secretion was measured in Krebs-Ringer-bicarbonate buffer containing 120 mM NaCl, 5 mM KCl, 2.5 mM CaCl2, 1.1 mM MgCl2, 25 mM NaHCO3 and 0.1% bovine serum albumin at 37° C. Inhibitors of insulin secretion (nifedipine and diazoxide) were added to buffer during preincubation (30 min) and throughout the incubation period. For determination of total cellular insulin content, insulin was extracted from cells with acid ethanol (10% glacial acetic acid in absolute ethanol) overnight at 4° C., followed by cell sonication. Total cellular and secreted insulin was assayed using insulin ELISA kit (ALPCO, Windham, N.H.). Protein concentrations were determined using DC protein assay system (Bio-Rad, Hercules, Calif.).

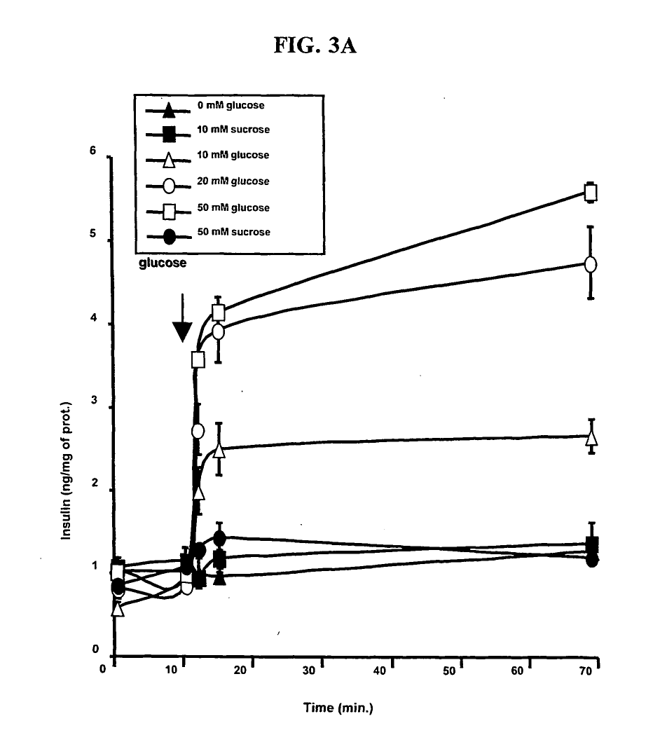

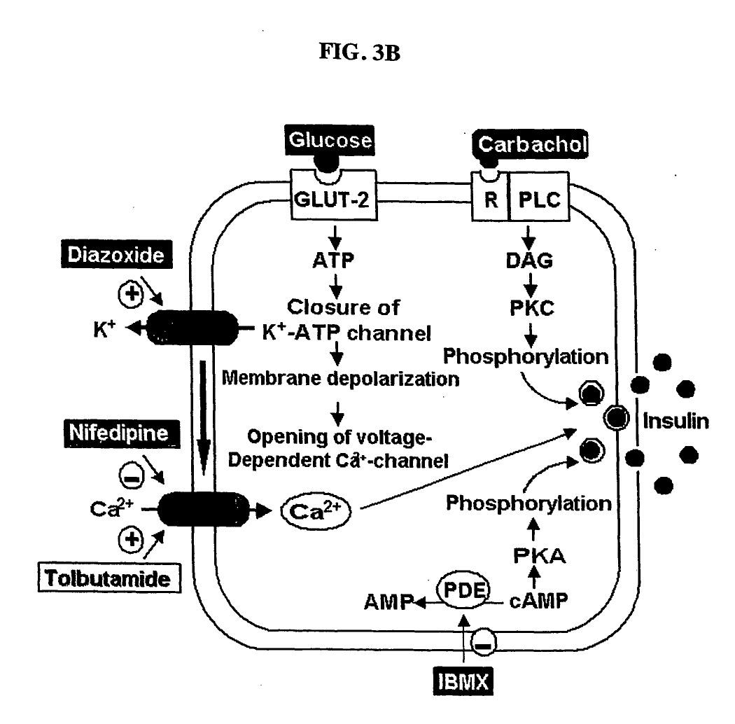

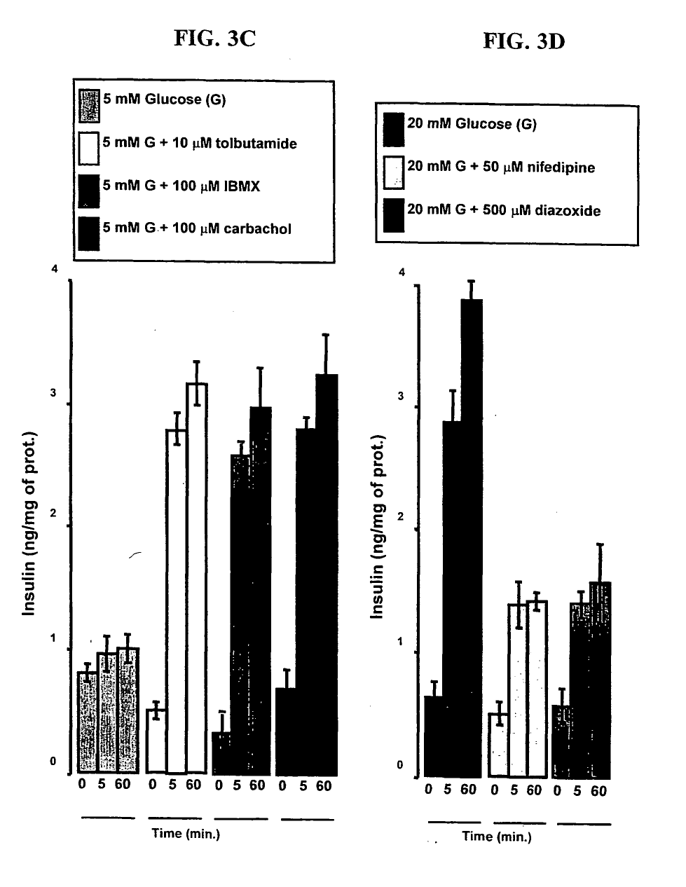

At the end of stage 5 the cells release insulin in response to glucose in a dose-dependent manner (FIG. 3A). Similar dose response curves have been observed in primary pancreatic islets in vitro (Csemus et al., Cell. Mol. Life Sci., 54:733, 1998). Comparison of insulin content and of insulin release at the end of stages 4 and 5 (see Table 1, below) showed that insulin-secreting islet clusters undergo progressive maturation during stage 5 with total insulin content of the cells increasing 5-fold and glucose-stimulated insulin release increasing more that 40-fold between stages 4 and 5.

| | | | | Glucose- |
|---|---|---|---|---|
| | | | | induced |
| | | Intracellular | Glucose- | insulin |
| | Protein | insulin | induced | release |
| | content | content | insulin release | (% of insulin |
| | (mg/well) | (ng/mg prot.) | (ng/mg prot.)* | content)* |
| 6 days of | 128 ± 9 | 28 ± 3 | 0.07 ± 0.08 | 0.25 ± 0.27 |
| expansion | | | | |
| 6 days of | 310 ± 24 | 145 ± 9 | 2.87 ± 0.10 | 1.98 ± 0.07 |
| differentiation | | | | |

*insulin released within 5 minutes in response to a 20 mM glucose stimulation.

Table 1. ES cells progressively differentiate to store and release insulin. Shown are properties of the cells at the end of the expansion and differentiation stages. Glucose-induced insulin release data correspond to the amount of insulin secreted within five minutes following 20 mM glucose stimulation. Data presented are means±SEM of the triplicate wells of the same ES cell culture. The results were reproduced in three independent experiments.

To determine if the islet clusters utilize physiological glucose-mediated signaling pathways, the effect of several well-characterized agonists and antagonists of insulin secretion were examined. The mechanism by which glucose stimulates insulin secretion in vivo is complex. As outlined in FIG. 3B, transport of glucose into the cell, and its metabolism results in ATP production, an event which, in turn, leads to inhibition of ATP-dependent K⁺ channels, cell membrane depolarization, opening of the voltage-dependent Ca⁺⁺ channels, and influx of extracellular Ca⁺⁺ into the cell. Additionally, intracellular Ca⁺⁺ can be elevated by release of Ca⁺⁺ from intracellular stores through other mechanisms. Elevation of free intracellular Ca⁺⁺ is coupled to multiple phosphorylation events modulated by protein kinase C (PKC) and protein kinase A (PKA) cascades, which ultimately lead to release of insulin from the cell (McClenaghan et al., J Mol. Med., 77:235, 1999).

The results of the effect of the agonists and antagonists on insulin secretion are shown in FIGS. 3C and D. All the agonists tested, a sulfonylurea inhibitor of ATP-dependent K+Channel (tolbutamide, Trube et al., Pflugers Arch., 407:493, 1986), an inhibitor of cyclic-AMP (cAMP) phosphodiesterase (3-isobutil-1-methylxanthine, IBMX, Montague et al., Biochem. J, 122:115, 1971), and an agonist of muscarinic cholinergic receptors (carbachol, Ahren et al., Prog. Brain Res., 84:209, 1990) stimulated insulin secretion in the presence of low concentration (5 mM) of glucose. Conversely, the antagonists sulfonamide, a diazoxide activator of ATP-dependent K+channel (Trube et al., Pflugers Arch., 407:493, 1986) and nifedipine, a blocker of L-type Ca⁺⁺ channel, one of the Ca⁺⁺ channels present in β-cells (Rojas et al., FEBS Lett., 26:265, 1990), inhibited insulin secretion in the presence of high glucose concentrations (20 mM). These results indicate that nomial pancreatic machinery is utilized for glucose-mediated insulin release.

### Example 5

### Grafting of Insulin-Producing Cells into Animal Models

Insulin cell clusters after 6 days of differentiation in vitro were dislodged from tissue culture plastic with trypsin or with EDTA, suspended in culturing medium, and grafted subcutaneously into streptozotocin induced diabetic mice. Clusters of islets were dislodged from the tissue culture plastic. Animals were injected subcutaneously between the shoulder blades or adjacent to the rib cage with the contents of one 6 cm confluent plate per animal, or about three to five million cells. Alternative routes of administration are injection into the portal vein, under the kidney capsule, or into the spleen.

In these experiments survival of insulin producing cells and vascularization of the grafts was examined. The analysis was carried out two and six weeks after cell transplantation. Extensive vascularizarion of the grafts was found, as well as good insulin cell survival at both time points.

The diabetic animals that received the cell grafts survived without extensive weight loss six weeks after transplantation (they were sacrificed at 6 weeks for the purpose of the analysis). All mock transplanted animals died within four weeks after mock transplantation. In order to assess the glycemic state of the transplanted animals, the amount of glucose in the blood of the animals is determined. Specifically, a glucometer is used to measure the amount of glucose in the blood. A normal mammal has a glucose level of about 90 mg/dl to about 150 mg/dl glucose, whereas a diabetic animal has a glucose level of about 200 mg/dl to about 600 mg/dl. Transplantation of cell grafts corrects the amount of glucose found in the blood to the normal level.

The insulin cell cultures can also be transfected with a gene of interest. In this embodiment, transformation is performed prior to transplantation. An example of a gene of interest is PDX-1.

In another embodiment, pancreatic precursor cells at different stages of differentiation are introduced into embryonic or adult animals to study the proliferation, survival and differentiation, in vivo.

Insulin cell clusters after 6 days of differentiation in vitro are dislodged from tissue culture plastic with trypsin or with EDTA, suspended in culturing medium, and grafted subcutaneously into diabetic or non-embryonic animals. The animals are either adult animals or embryos. For introduction into adult animals, clusters of islets are dislodged from the tissue culture plastic. The cells are introduced into adult animals as described below. For introduction into embryos, clusters of pancreatic endocrine cells can be introduced in utero and the development of the cells is monitored (Pschera et al., J. Perinatal. Med. 28:346-54, 2000).

### Example 6

### Dissociation and Re-Association of Insulin Cell Clusters

Native dissociated pancreatic islets can re-associate to form three-dimensional aggregates with normal islet architecture (Halban et al., Diabetes, 36, 783-90, 1987). The capacity of the ES cell-derived insulin clusters to form similar aggregates was investigated. The cell clusters after 7 days of differentiation were dislodged from the tissue culture plastic in physiological buffer in the absence of calcium and in the presence of EDTA, and individual cells were obtained by passing the clusters through a hypodermic needle. The cells were allowed to aggregate in suspension for various amounts of time. Secondary cell aggregates form readily from the individual cells with the kinetics and the aggregate morphology similar to that of the native pancreatic islet cells. These clusters are useful for grafting in vivo and for the investigation of the mechanism of pancreatic islet morphogenesis.

The results described herein demonstrate that ES cells can generate endocrine progenitor cells that proliferate and differentiate into cells with high insulin content. When exposed to glucose, these cells release insulin with the fast kinetics utilizing physiologically relevant mechanisms. Importantly, insulin- and other hormone-producing endocrine cells that are generated in this system, self-assemble into structures with the morphological and functional characteristics of normal pancreatic islets. This advance may be of particular importance for several reasons. First, it provides an accessible model system to study early endocrine progenitor cells that are difficult or impossible to obtain in vivo as well as to study morphogenesis of pancreatic islet. Second, this ES cell system allows routine production of insulin-secreting cells in the context of the other islet cell types known to play important role in regulation of insulin secretion (Ahren, Diabetologia, 43:393, 2000; Soria et al., Pflugers Arch., 440:1, 2000). The self-assembly of distinct cell types into the organized structures provides a powerful system to analyze the mechanisms relevant to fine control of glucose homeostasis. Third, this differentiation system, when applied to human ES cells, provides an unlimited source of functional pancreatic islets for treatment of type I, as well as type II diabetes, where insulin resistance is usually followed by declining β-cell function and insulin deficiency (Hamman et al., Diabetes Metab. Rev., 8:287, 1992). Recent work suggests that pancreatic islets obtained from cadavers can function in the liver after grafting into portal vein (Shapiro et al., N. Engl. J. Med., 27:230, 2000). However, wide application of islet grafting is limited by the availability of suitable tissue, and by immunological rejection of the graft. Because ES cells can be genetically manipulated to reduce, or eliminate the problem of rejection, they hold great promise as a source of large numbers of immunologically compatible pancreatic islets.

### Example 7

### Use of Pancreatic Endocrine Cells Differentiated from ES Cells in a Bioartificial Pancreas

There is a need to provide a biocompatible and implantable device containing islets of Langerhans, or the insulin producing β cells, that can supply the hormone insulin for the purpose of controlling blood glucose levels in people with diabetes mellitus requiring insulin. Insufficient regulation of blood glucose levels in people with diabetes has been associated with the development of long-term health problems such as kidney disease, blindness, coronary artery disease, stroke, and gangrene resulting in amputation. Therefore, there is a need to replace conventional insulin injections with a device that can provide more precise control of blood glucose levels.

Many modalities are currently available to replace the impaired pancreatic beta cell function in diabetes mellitus patients. The electromechanical modality utilizes insulin delivery systems that release insulin in response to blood glucose levels that are continuously measured via a glucose sensor. Difficulties with the sensors led to the development of programmed insulin delivery via a continuous perfusion pump. This approach however also falls short of the in vivo regulation, i.e. the regulation of insulin secretion by glucose and its modulation by several hormonal and neuronal factors.

Pancreas transplants are another approach (for example see Shapiro et al., N Engl. J. Med. 343(4):230-8, 2000). Unfortunately, this approach suffers from limited availability of transplantable tissue and immune rejection.

To overcome these problems, bioartificial pancreases have been developed. These systems separate the transplanted tissue from the diabetic recipient by an artificial barrier, which diminishes immune rejection, yet allows the transfer of the glycemic signal from the blood to the islet cells and the transfer of the pancreatic hormones from the islet cells to the blood. An artificial pancreas accomplishes this by having a selectively permeable barrier, which is permeable to glucose and insulin, but not to immunoglobulins and immunocytes. Artificial pancreas devices work based on the transfer through the membrane of a glycemic signal from blood to the pancreatic endocrine cells, and insulin from the pancreatic endocrine cells to the recipient. In one embodiment, the pancreatic endocrine cells are in the form of islets.

In general, the transfer of a substance from one compartment to the other across a membrane can be achieved either by diffusion, dialysis, or by convection, ultrafiltration or a combination of these methods. Artificial pancreases are generally divided among those that utilize diffusion mechanisms, those that utilize convection mechanisms, or those that utilize a combination of both mechanisms. Diffusion represents the transfer of the substance itself without transfer of the solvent. Convection, in contrast, involves the transfer of the solvent and any molecules dissolved therein as long as they are smaller than the pores of the membrane.

Suitable devices for use with pancreatic endocrine cells as an artificial pancreas are well known in the art. Specific, non-limiting examples devices of use are disclosed in U.S. Pat. No. 5,741,334; U.S. Pat. No. 5,702,444; U.S. Pat. No. 5,855,616; U.S. Pat. No. 5,913,998; U.S. Pat. Nos. 6,023,009; and 6,165,225, all of which are incorporated by reference herein.

Thus, the methods disclosed herein can be used to generate pancreatic endocrine cells, artificial islets differentiated from ES cells, or re-aggregated pancreatic endocrine cells differentiated form ES cells. These cells are then included in a device as a bioartificial pancreas, and the bioartificial pancreas is then implanted into a subject. The implantation of the bioartificial pancreas results in the treatment of a disorder. In embodiment, the implantation of the bioartificial pancreas results in the treatment of diabetes.

### Example 8

### Use of LIF to Regulate the Differentiation of ES Cultures

Transcription factor PDX-1 plays a critical role in pancreatic development and is an essential component of an adult endocrine pancreatic gene expression machinery (see Ahlgren et al. Development 122(5):1409-16, 1996; Jonsson et al., Nature. 371(6498):606-9, 1994). In addition the transcription factor engrailed-1 (EN-1) is one the primary regulators of neural development in CNS (Simon et al., J. Neurosci. 21:3126-3134, 2001).

The methods disclosed herein include five stages: (1) expansion of ES cells (2) generation of EB (3) selection for CNS precursor cells (4) expansion of pancreatic (versus central nervous system (CNS)) precursor cells, and (5) differentiation of pancreatic endocrine cells (versus differentiation of neuronal cells). Expansion of ES cells and generation of EB was performed as disclosed herein. EB were cultured in DMEM/15% serum (ES medium) with LIF (1000 units (U)/ml) for 4 days with changing medium every 2 day. After 4 days, EBs were transferred to a tissue culture dish cultured in ITS medium containing fibronectin for 10-12 days. EBs which were kept in absence of LIF in stage II were phenotypically different than EB cultured in the presence of LIF in stage II. In stage 1V, ES-derived CNS precursor were cultured in N2 medium in the presence of bFGF (20 ng/ml) and Shh (500 ng/ml) and FGF8 (100 ng/ml) for 4 days and after withdrawal of bFGF/SHH/FGF8, differentiated them for 10-12 day in N2 medium with ascorbic acid. Specifically, EB cultured in the absence of LIF were spread out in stage III. EBs which were treated with LIF maintained a round shape and CNS precursor cells migrated from attaching point of EB in dishes. Therefore, a selection for CNS precursor was accomplished by culturing in the presence of LIF.

Treatment of ES cell cultures with LIF at stage 2 (EB formation) increases the expression of EN-1 at stage 4. Specifically, up to 80% of the total ES cell-derived cell population becomes EN-1 positive at stage 4 if LIF is present at stage 2. As a result of this treatment, the overall yield of neurons at stage 5 is also increased. Only few PDX-1 positive cells are generated under these conditions.

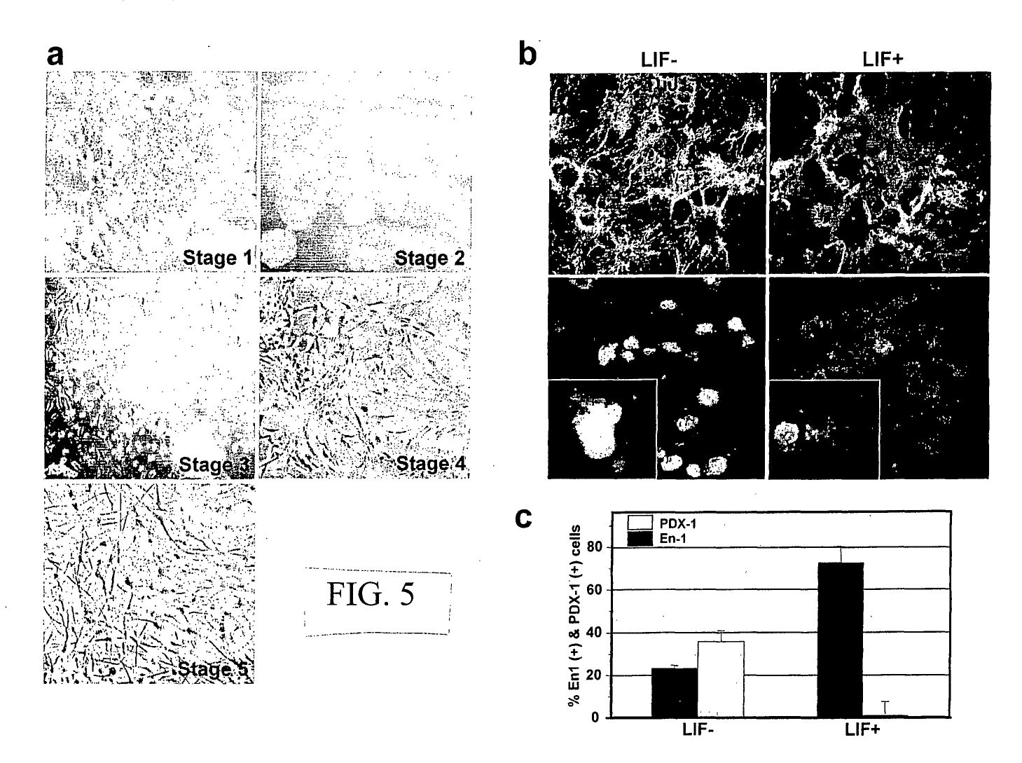

Conversely, if LIF is not included in the ES cell cultures at stage 2, or if a very low concentration of LIF is included, the number of EN-1 cells at stage 4 is drastically reduced, whereas the number of PDX-1 positive cells is increased (see FIG. 5). These experiments demonstrate that LIF treatment can be used to control the developmental fate of the ES cell cultures. Thus, in one embodiment, the absence of LIF at stage 2 increases the production of PDX+ progenitors of insulin producing cells. In several embodiments, the ES cultures are treated with less than 500 U/ml of exogenously added LIF, or less than 200 U/ml of exogenously added LIF, or less than 100 U/ml, exogenously added LIF, or less than 50 U/ml of exogenously added LIF, or less than 10 U/ml of exogenously added LIF, or less than 1 U/ml of exogenously added LIF, or in the absence of LIF at stage 2 in order to generate insulin-producing cells.

### REFERENCES

1. J. A. Thomson, J. Itskovitz-Eldor, S. S. Shapiro, M. A. Waknitz, J. J. Swiergiel, V. S. Marshall, J. M. Jones, Science 282, 5391 (1998)

2. M. J. Shamblot, J. Axelman, S. Wang, E. M. Bugg, J. M. Littlefield, P. J. Donovan, P. D. Blumenthal, G. R. Huggins, J. D. Gearhart, Proc. Natl. Acad. Sci. USA 95, 13726 (1998)

3. G. M. Keller, Curr. Opin. Cell. Biol. 7, 862 (1995)

4. J. Rohwedel, K. Guan, A. M. Wobus, Cells Tiss. Organs 165, 190 (1999)

5. M. G. Klug, M. H. Soonpaa, J. I. Koh, L. J. Field, J. Clin. Invest. 98, 216 (1996)

6. S. Okabe, K. Forsberg-Nilsson, A. C. Spiro, M. Segal, R. D. McKay, Mech. Dev. 59, 89 (1996)

7. O. Brustle, K. N. Jones, R. D. Learish, K. Karram, K. Choudhary, O. D. Wiestler, I. D. Duncan, R. D. McKay, Science 285, 754 (1999)

8. J. M. Slack, Development 121, 1569 (1995)

9. Placzek M, Furley A, Curr. Biol.6, 526 (1996)

10. S. K. Kim, M. Hebrok, D. A. Melton, Development 124, 4243 (1997)

11. U. Ahlgren, S. L. Pfaff, T. M. Jessell, T. Edlund, H. Edlund, Nature 16, 257 (1997)

12. L. St-Onge, B. Sosa-Pineda, K Chowdhury, A. Mansouri, P. Gruss, Nature 22, 406 (1997)

13. F. G. Naya, H. P. Huang, Y. Qiu, H. Mutoh, F. G. DeMayo, A. B. Leiter, M. J. Tsai, Genes Dev. 15, 2323 (1997)

14. V. M. Schwitzgebel, D. W. Scheel, J. R. Conners, J. Kalamaras, J. E. Lee, D. J. Anderson, L. Sussel, J. D. Johnson, M. S. German, Development, 127, 3533 (2000)

15. H. Li, S. Arber, T. M. Jessell, H. Edlund, Nat. Genet.23, 67 (1999)

16. S. H. Lee, N. Lumelsky, L. Studer, J. M. Auerbach, R. McKay, Nat. Biotechnol. 18, 675 (2000)

17. K. Frederiksen, R. D. McKay, J. Neurosci. 8, 1144 (1988)

18. U. Lendahl, L. B. Zimmerman, R. D. McKay, Cell 23, 585 (1990)

19. E. Hunziker, M. Stein, Biochem. Biophys. Res. Commun. 29, 116 (2000)

20. G. J. Brewer, J. R. Torricelli, E. K. Evege, P. J. Price, J. Neurosci. Res. 35, 567, (1993)

21. T. Otonkoski, T. M. Beattie, M. I. Mally, C. Ricordi, A. J. Hayek, J. Clin. Invest. 92, 1459 (1993)

22. B. M. Wentworth, I. M. Schaefer, L. Villa-Komaroff, J. M. Chirgwin, J. Mol. Evol. 23, 305 (1986)

23. M. E. Rothenberg, C. D. Eilertson, K. Klein, Y. Zhou, I. Lindberg, J. K. McDonald, R. B. Mackin, B. D. Noe, J. Biol. Chem. 270, 10136 (1995)

24. K. Ekawa, M. Nishi, S. Ohagi, T. Sanke, K. J. Nanjo, Mol. Endocrinol. 19, 79 (1997)

25. G. Waeber, N. Thompson, J. A. Haefliger, P. J. Nicod, J. Biol. Chem. 28, 26912 (1994)

26. H. Ohlsson, K. Karlsson, T. Edlund, EMBO J. 12, 4251 (1993)

27. Y. Guz, M. R. Montminy, R. Stein, J. Leonard, L. W. Gamer, C. V. Wright, G. Teitelman, Development 121, 11 (1995)

28. A. Ferreira, A. Caceres, J. Neurosci. Res. 32, 516 (1992)

29. K. Hadjantonakis, M. Gertsenstein, M. Ikawa, M. Okabe, A. Nagy, Mech. Dev. 76, 79 (1998)

30. W. W. Kao, C. Y. Liu, R. L. Converse, A. Shiraishi, C. W. Kao, M. Ishizaki, T. Doetschman, J. Duffy, Ophthalmol. Vis. Sci. 37, 2572 (1996)

31. A. Vinik, R. Rafaeloff, G. Pittenger, L. Rosenberg, W. Duguid, Horm. Metab. Res. 29, 278 (1997)

32. V. J. Csernus, T. Hammer, D. Peschke, E. Peschke, Cell. Mol. Life Sci. 54, 733 (1998)

33. N. H. McClenaghan, P. R. Flatt, J. Mol. Med. 77, 235 (1999)

34. G. Trube, P. Rorsman, T. Ohno-Shosaku, Pflugers Arch. 407, 493 (1986)

35. W. Montague, J. R. Cook, Biochem. J. 122, 115 (1971)

36. B. Ahren, S. Karlsson, S. Lindskog, Prog. Brain Res. 84, 209 (1990)

37. E. Rojas, J. Hidalgo, P. B. Carroll, M. X. Li, I. Atwater, FEBS Lett. 26, 265 (1990)

38. B. Soria, E. Roche, G. Bema, T. Leon-Quinto, J. A. Reig, F. Martin, Diabetes 49, 157 (2000)

39. B. Ahren, Diabetologia 43, 393 (2000)

40. B. Soria, E. Andreu, G. Berna, E. Fuentes, A. Gil, T. Leon-Quinto, F. Martin, E. Montanya, A. Nadal, J. A. Reig, C. Ripoll, E. Roche, J. V. Sanchez-Andres, J. Segura, Pflugers Arch. 440, 1 (2000)

41. R. F. Hamman, Diabetes Metab. Rev. 8, 287 (1992)

42. A. M. Shapiro, J. R. Lakey, E. A. Ryan, G. S. Korbutt, E. Toth, G. L. Warnock, N. M. Kneteman, R. V. Rajotte, N. Engl. J. Med. 343, 230 (2000)

43. A. M. Shapiro J. R. Lakey, E. A. Ryan, G. S. Korbuff, E. Toth, G. L. Warnock, N. M. Kneteman, R. V. Rajotte N Engl. J. Med. 343, 289 (2000).

44. H. H. Simon, H. Saueressig, W. Wurst., M. Goulding, O'Leary, D. D. M., J. Neurosci. 21: 3126-3134, 2001.

In view of the many possible embodiments to which the principles of our invention may be applied, it should be recognized that the illustrated embodiment is only a preferred example of the invention and should not be taken as a limitation on the scope of the invention. Rather, the scope of the invention is defined by the following claims. We therefore claim as our invention all that comes within the scope and spirit of these claims.

We claim:

1. An isolated pancreatic endocrine cell, wherein said cell is differentiated from an embryonic stem cell in vitro, and wherein said cell secretes a pancreatic hormone.

2. The isolated pancreatic endocrine cell of claim 1, wherein the pancreatic endocrine cell comprises a β-cell, an α-cell, a δ-cell, or a PP cell, or combinations thereof.

3. The isolated pancreatic endocrine cell of claim 1, wherein the pancreatic endocrine cell is a β-cell.

4. The isolated pancreatic endocrine cell of claim 1, wherein the pancreatic endocrine cell is a murine cell.

5. The isolated pancreatic endocrine cell of claim 1, wherein the pancreatic endocrine cell is a human cell.

6. The isolated pancreatic endocrine cell of claim 1, wherein the pancreatic hormone is insulin, glucagon, somatostatin, or pancreatic polypeptide.

7. A method for differentiating embryonic stem cells to endocrine cells, comprising
selecting endocrine precursor cells from embryonic stem cells or from embryoid bodies differentiated from embryonic stem cells;
expanding the endocrine precursor cells by culturing endocrine cells in an expansion medium that comprises a growth factor; and
differentiating the expanded endocrine precursor cells in a differentiation medium to differentiated endocrine cells.

8. The method of claim 7, wherein the selection of endocrine precursor cells comprises selecting cells that express nestin.

9. The method of claim 7, wherein the expansion medium is N2 medium containing B27 media supplement.

10. The method of claim 7, wherein the growth factor is bFGF.

11. The method of claim 7, wherein the differentiation medium comprises N2 medium containing B27 media and nicotinamide in the absence of the growth factor.

12. The method of claim 7, wherein the endocrine cells secrete insulin, glucagon, somatostatin, pancreatic polypeptide, or a combination thereof.

13. The method of claim 7, wherein the embryonic stem cells comprise murine, procine, or human embryonic stem cells.

14. The method of claim 13, wherein the embryonic stem cells are human embryonic stem cells.

15. The method of claim 7, wherein the endocrine cells are pancreatic endocrine cells.

16. The method of claim 15, wherein the pancreatic endocrine cells comprises a β-cell, an α-cell, a δ-cell or a PP cell, or a combination thereof.

17. The method of claim 7, wherein the endocrine precursor cells are selected from embryoid bodies.

18. The method of claim 7, wherein the generation of embryoid bodies comprises culturing expanded undifferentiated embryonic stem cells in suspension.

19. The method of claim 7, wherein the step of culturing the embryoid bodies to select endocrine precursor cells comprises culturing the embryoid bodies in a serum-free medium.

20. The method of claim 7, wherein the step of culturing the embryoid bodies to select for endocrine precursor cells comprises culturing the embryoid bodies on a fibronectin-coated surface.

21. The method of claim 7, wherein the step of culturing the embryoid bodies to select for endocrine precursor cells comprises culturing the embryoid bodies for about 6 to about 8 days.

22. The method of claim 7, further comprising aggregating the differentiated endocrine cells.

23. A differentiated endocrine cell produced by the method of claim 7.

24. The endocrine cell of claim 23, wherein the endocrine cell is a pancreatic endocrine cell.

25. The endocrine cell of claim 23, wherein the pancreatic endocrine cell secrete insulin, glucagon, somatostatin, pancreatic polypeptide, or a combination thereof.

26. An artificial islet of Langerhans comprising the pancreatic endocrine cell produced by the method of claim 23.

27. A method of producing an artificial islet of Langerhans, comprising
generating embryoid bodies from a culture of undifferentiated embryonic stem cells;
selecting pancreatic endocrine precursor cells;
expanding the pancreatic endocrine precursor cells by culturing pancreatic endocrine cells in an expansion medium that comprises a growth factor; and
differentiating the expanded pancreatic endocrine precursor cells in a differentiation mediun to form pancreatic endocrine cells, and wherein the differentiation produces the artificial islet.

28. The method of claim 27, wherein the selection of endocrine precursor cells comprises selecting cells that express nestin.

29. The method of claim 27, wherein the expansion expansion medium is N2 medium containing B27 media supplement.

30. The method of claim 27, wherein the growth factor is bFGF.

31. The method of claim 27, wherein the differentiation medium comprises N2 medium containing B27 medium in the absence of the growth factor.

32. The method of claim 27, wherein the endocrine cells secrete insulin.

32. The method of claim 27, wherein the endocrine cells secrete glucagon, somatostatin, pancreatic polypeptide, or a combination thereof.

33. The method of claim 27, wherein the embryonic stem cells are murine, procine, or human embryonic stem cells.

34. The method of claim 27, wherein the embryonic stem cells are human embryonic stem cells.

35. The method of claim 27, wherein the generation of embryoid bodies comprises culturing expanded embryonic stem cells for about 4 to about 7 days.

36. The method of claim 27, wherein the generation of embryoid bodies comprises culturing expanded undifferentiated embryonic stem cells in suspension.

37. The method of claim 27, wherein the step of culturing the embryoid bodies to select endocrine precursor cells comprises culturing the embryoid bodies in a serum-free medium.

38. The method of claim 27, wherein the step of culturing the embryoid bodies to select for endocrine precursor cells comprises culturing the embryoid bodies on a fibronectin-coated surface.

39. The method of claim 27, wherein the step of culturing the embryoid bodies to select for endocrine precursor cells comprises culturing the embryoid bodies for about 6 to about 8 days.

40. A method for testing an agent to determine the effect of the agent on secretion or expression of a pancreatic hormone, comprising:
contacting pancreatic endocrine cells with the agent, wherein the pancreatic endocrine cells are differentiated from embryonic stem cells; and
assaying a parameter of the pancreatic endocrine cell to determine the effect of the agent on the secretion or expression of the pancreatic hormone.

41. The method of claim 40, wherein the pancreatic endocrine hormone is insulin.

42. A method of enhancing insulin production in a subject, comprising:
administering to the subject a therapeutically effective amount of a pancreatic endocrine cell produced by the method of claim 7.

43. The method of claim 42, wherein the subject is a diabetic.

44. A pharmacological composition comprising
a pancreatic endocrine cell produced by the method of claim 7; and
a pharmacologically acceptable carrier.

45. The method of claim 7, wherein selecting endocrine precursor cells from embryonic stem cells or from embryoid bodies differentiated from embryonic stem cells is performed in the absence of exogenously added LIF.

46. The method of claim 27, wherein selecting pancreatic endocrine precursor cells includes culturing embryoid bodies in the absence of exogenously added LIF.


SEQUENCE LISTING (18 sequences)

SEQ ID NO: 1 - DNA, 22 bp, Artificial Sequence
  Oligonucleotide primer
tagtgaccagctataatcagag

SEQ ID NO: 2 - DNA, 20 bp, Artificial Sequence
  Oligonucleotide primer
acgccaaggtctgaaggtcc

SEQ ID NO: 3 - DNA, 19 bp, Artificial Sequence
  Oligonucleotide primer
ccctgctggccctgctctt

SEQ ID NO: 4 - DNA, 20 bp, Artificial Sequence
  Oligonucleotide primer
aggtctgaaggtcacctgct

SEQ ID NO: 5 - DNA, 19 bp, Artificial Sequence
  Oligonucleotide primer
tcatgacgtttggcaagtt

SEQ ID NO: 6 - DNA, 20 bp, Artificial Sequence
  Oligonucleotide primer
cagaggagaaccccagatca

SEQ ID NO: 7 - DNA, 20 bp, Artificial Sequence
  Oligonucleotide primer
gattccctatttggatcccc

SEQ ID NO: 8 - DNA, 20 bp, Artificial Sequence
  Oligonucleotide primer
ctctctgtggcactgaacca

SEQ ID NO: 9 - DNA, 19 bp, Artificial Sequence
  Oligonucleotide primer
agcttttctttgccctgac

SEQ ID NO: 10 - DNA, 20 bp, Artificial Sequence
  Oligonucleotide primer
ccctgggatgaagaggagac

SEQ ID NO: 11 - DNA, 20 bp, Artificial Sequence
  Oligonucleotide primer
tgtaggcagtacgggtcctc

SEQ ID NO: 12 - DNA, 20 bp, Artificial Sequence
  Oligonucleotide primer
ccaccccagtttacaagctc

SEQ ID NO: 13 - DNA, 20 bp, Artificial Sequence
  Oligonucleotide primer
cattgttgcaccttgtcacc

SEQ ID NO: 14 - DNA, 20 bp, Artificial Sequence
  Oligonucleotide primer
ttctgctgctttccctcatt

SEQ ID NO: 15 - DNA, 20 bp, Artificial Sequence
  Oligonucleotide primer
gcaaatgtgtgtttgatgcc

SEQ ID NO: 16 - DNA, 20 bp, Artificial Sequence
  Oligonucleotide primer
atgaccaaactcttggaccg

SEQ ID NO: 17 - DNA, 19 bp, Artificial Sequence
  Oligonucleotide primer
atggatgacgatatcgctg

SEQ ID NO: 18 - DNA, 19 bp, Artificial Sequence
  Oligonucleotide primer
atgaggtagtctgtcaggt